SEQUENCE LISTING (2 sequences)

SEQ ID NO: 1 - protein, 708 aa, Bos taurus
MKLFVPALLSLGALGLCLAAPRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 2 - protein, 710 aa, Homo sapiens
MKLVFLVLLFLGALGLCLAGRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

(19) United States
(12) Patent Application Publication
(10) Pub. No.: US 2026/0344693 A1
(43) Pub. Date: Nov. 5, 2026

(54) LACTOFERRIN COMPLEXES, COMPOSITIONS COMPRISING THE SAME, AND METHODS OF MAKING AND USING THE SAME

(71) Applicants: Dairy Management Inc., Rosemont, IL (US); Cornell University, Ithaca, NY (US)

(72) Inventors: Tiantian Lin, Ithaca, NY (US); Younas Dadmohammadi, Ithaca, NY (US); Alireza Abbaspourrad, Ithaca, NY (US); Yufeng Zhou, Ithaca, NY (US); Hari Meletharayil, Palatine, IL (US); Emil S. Nashed, North Haledon, NJ (US); Rohit Kapoor, Elk Grove Village, IL (US)

(21) Appl. No.: 19/121,517

(22) PCT Filed: Oct. 20, 2023
(86) PCT No.: PCT/US2023/077376

§ 371 Date: Apr. 16, 2025

Related U.S. Application Data
(60) Provisional application No. 63/380,388, filed on Oct. 21, 2022.

(51) Int. Cl. A23C 21/06; A23L 33/19; C07K 14/79

(52) U.S. Cl. CPC A23C 21/06; A23L 33/19; C07K 14/79

(57) ABSTRACT

Described herein are complexes comprising a lactoferrin and a whey protein hydrolysate, compositions comprising the same, and methods of making and using the same. Compositions of the present invention may comprise a complex comprising a lactoferrin and a whey protein hydrolysate, and may be an aqueous composition.

### STATEMENT REGARDING ELECTRONIC FILING OF A SEQUENCE LISTING

A Sequence Listing in XML format, entitled 1213-7WO_ST26.xml, 4,100 bytes in size, generated on Oct. 17, 2023, and filed herewith, is hereby incorporated by reference in its entirety for its disclosures.

### FIELD

The present invention relates to complexes comprising a lactoferrin and a whey protein hydrolysate, compositions comprising such complexes, and methods of making and using such complexes.

### BACKGROUND

Lactoferrin (LF) is an iron-binding multifunctional protein occurring in many biological secretions, including milk. It possesses iron binding/transferring, antibacterial, anti-inflammatory, and anti-carcinogenic properties. It promotes cell growth and detoxifies harmful free radicals and has anti-bacterial, anti-viral, anti-inflammatory, and anti-carcinogenic properties. Because of the multiple biological functions of LF, it has been incorporated into many commercialized products, including infant formulas, nutritional supplements, therapeutic drinks, and cosmetics. However, LF is sensitive to denaturation induced by thermal processing, especially under neutral pH conditions, which causes structural changes and the loss of biological functionality.

### SUMMARY

A first aspect of the present invention is directed to a complex comprising: a lactoferrin and a whey protein hydrolysate, wherein the lactoferrin and the whey protein hydrolysate are associated via electrostatic interactions.

An additional aspect of the present invention is directed to a composition comprising a complex of the present invention. In some embodiments, the composition is an aqueous composition.

A further aspect of the present invention is directed to a method of preparing a complex of the present invention, the method comprising: providing a composition comprising a lactoferrin and a whey protein hydrolysate at a pH in a range of about 5, 5.5, or 5.8 to about 6, 6.5, or 7, and mixing the composition, thereby providing the complex.

A further aspect of the present invention is directed to an article comprising a complex of the present invention, a composition of the present invention, and/or a complex prepared according to a method of the present invention. In some embodiments, the article is a food product (e.g., infant formula, a dairy product, etc.), nutritional supplement, therapeutic drink, and/or cosmetic.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1 shows a schematic depicting an exemplary method for preparing a binary complex of the present invention using a lactoferrin (LF) and a whey protein hydrolysate (WPH) according to some embodiments of the present invention.

FIGS. 2-5 are graphs depicting the effects of pH and mixing ratio on the turbidity of lactoferrin and whey protein hydrolysate mixtures.

FIGS. 6-9 are graphs depicting the effects of pH and mixing ratio on the mean particle size of complexes obtained from lactoferrin and whey protein hydrolysate mixtures.

FIGS. 10-15 are graphs depicting the effects of mixing ratio on the particle size distribution of lactoferrin, whey protein hydrolysate, or mixtures thereof at pH 5.8.

FIGS. 16-19 are graphs depicting the effect of pH and mixing ratio on the zeta potential of lactoferrin and whey protein hydrolysate mixtures.

FIGS. 20-21 show the effect of concentration (1%-10% w/v), mixing temperature (45 °C), and time (0, 10, 20, or 30 minutes) on the turbidity of LF-WPH mixtures at ratios of 1:1 and 1:2 (LF:WPH).

FIGS. 22-23 show the effect of concentration (1%-10% w/v), mixing temperature (45 °C), and time (0, 10, 20, or 30 minutes) on the mean particle size of LF-WPH mixtures at ratios of 1:1 and 1:2 (LF:WPH).

FIGS. 24-25 show the effect of concentration (1%-10% w/v), mixing temperature (45 °C), and time (0, 10, 20, or 30 minutes) on the zeta potential of LF-WPH mixtures at ratios of 1:1 and 1:2 (LF:WPH).

FIG. 26 shows optical microscopy and scan electron microscopy images of lactoferrin and whey protein hydrolysate mixture samples at pH 5.8 and different mixing ratios.

FIGS. 27-32 show Small Angle X-ray Scattering (SAXS) curves (FIG. 27) and corresponding pair distance distribution functions (PDDFs) (FIGS. 28-32) evaluated based on indirect Fourier transformation of lactoferrin, whey protein hydrolysate, and lactoferrin-whey protein hydrolysate complexes at pH 5.8 and mixing ratios of 2:1 and 1:1, along with radius of gyration (R_{g}) and largest intermolecular distance (Dₘₐₓ) values.

FIGS. 33-34 show optical microscopy images of the supernatant or sediment of lactoferrin and whey protein hydrolysate mixtures at pH 5.8 and lactoferrin:whey protein hydrolysate ratios of 2:1 (FIG. 33) and 1:1 (FIG. 34) after overnight setting at 4 °C.

FIG. 35 shows optical images of pure lactoferrin; centrifuged, freeze-dried lactoferrin/whey protein hydrolysate complex; and uncentrifuged, direct freeze-dried lactoferrin/whey protein hydrolysate complex samples in PBS (10 mM, pH 7) before and after thermal treatment.

FIG. 36 is a graph depicting turbidity of pure lactoferrin samples and centrifuged, freeze-dried lactoferrin/whey protein hydrolysate complex samples in PBS (10 mM, pH 7) before and after thermal treatment.

FIG. 37 is a graph depicting turbidity of pure lactoferrin samples and uncentrifuged, direct freeze-dried lactoferrin/whey protein hydrolysate complex samples in PBS (10 mM, pH 7) before and after thermal treatment.

FIG. 38 is a graph depicting mean particle size of pure lactoferrin samples and centrifuged, freeze-dried lactoferrin/whey protein hydrolysate complex samples in PBS (10 mM, pH 7) before and after thermal treatment.

FIG. 39 is a graph depicting mean particle size of pure lactoferrin samples and uncentrifuged, direct freeze-dried lactoferrin/whey protein hydrolysate complex samples in PBS (10 mM, pH 7) before and after thermal treatment.

FIG. 40 is a graph depicting lactoferrin loading ratio in pure lactoferrin samples and in centrifuged, freeze-dried lactoferrin/whey protein hydrolysate complex samples in PBS (10 mM, pH 7) before and after thermal treatment.

FIG. 41 is a graph depicting lactoferrin loading ratio in pure lactoferrin samples and in uncentrifuged, direct freeze-dried lactoferrin/whey protein hydrolysate complex samples in PBS (10 mM, pH 7) before and after thermal treatment.

FIG. 42 is a graph depicting lactoferrin retention percentage in pure lactoferrin samples and in centrifuged, freeze-dried lactoferrin/whey protein hydrolysate complex samples in PBS (10 mM, pH 7) before and after thermal treatment.

FIG. 43 is a graph depicting lactoferrin retention percentage in pure lactoferrin samples and in uncentrifuged, direct freeze-dried lactoferrin/whey protein hydrolysate complex samples in PBS (10 mM, pH 7) before and after thermal treatment.

FIG. 44 is an image of an SDS-PAGE gel with pure lactoferrin (LF) samples and centrifuged, freeze-dried lactoferrin/whey protein hydrolysate complex (LF-WPH) samples in PBS (10 mM, pH 7) before and after thermal treatment.

FIG. 45 is an image of an SDS-PAGE gel with pure lactoferrin (LF) samples and uncentrifuged, direct freeze-dried lactoferrin/whey protein hydrolysate complex (LF-WPH direct mix) samples in PBS (10 mM, pH 7) before and after thermal treatment.

FIG. 46 shows optical images of pure lactoferrin samples, centrifuged, freeze-dried lactoferrin-whey protein hydrolysate complex samples, and uncentrifuged, direct freeze-dried lactoferrin-whey protein hydrolysate complex samples in PBS (10 mM, pH 7) before and after oil bath heating treatment.

FIG. 47 is a graph depicting turbidity of pure lactoferrin samples and centrifuged, freeze-dried lactoferrin/whey protein hydrolysate complex samples in PBS (10 mM, pH 7) before and after oil bath heating treatment.

FIG. 48 is a graph depicting turbidity of pure lactoferrin samples and uncentrifuged, direct freeze-dried lactoferrin/whey protein hydrolysate complex samples in PBS (10 mM, pH 7) before and after oil bath heating treatment.

FIG. 49 is a graph depicting mean particle size of pure lactoferrin samples and centrifuged, freeze-dried lactoferrin/whey protein hydrolysate complex samples in PBS (10 mM, pH 7) before and after oil bath heating treatment.

FIG. 50 is a graph depicting mean particle size of pure lactoferrin samples and uncentrifuged, direct freeze-dried lactoferrin/whey protein hydrolysate complex samples in PBS (10 mM, pH 7) before and after oil bath heating treatment.

FIG. 51 is a graph depicting lactoferrin loading ratio in pure lactoferrin samples and in centrifuged, freeze-dried lactoferrin/whey protein hydrolysate complex samples in PBS (10 mM, pH 7) before and after oil bath heating treatment.

FIG. 52 is a graph depicting lactoferrin loading ratio in pure lactoferrin samples and in uncentrifuged, direct freeze-dried lactoferrin/whey protein hydrolysate complex samples in PBS (10 mM, pH 7) before and after oil bath heating treatment.

FIG. 53 is a graph depicting lactoferrin retention percentage in pure lactoferrin samples and in centrifuged, freeze-dried lactoferrin/whey protein hydrolysate complex samples in PBS (10 mM, pH 7) before and after oil bath heating treatment.

FIG. 54 is a graph depicting lactoferrin retention percentage in pure lactoferrin samples and in uncentrifuged, direct freeze-dried lactoferrin/whey protein hydrolysate complex samples in PBS (10 mM, pH 7) before and after oil bath heating treatment.

FIG. 55 is a graph depicting the growth of Staphylococcus aureus, as indicated by OD625 nm, at 37 °C in the presence of pure lactoferrin, pure whey protein hydrolysate, or lactoferrin/whey protein hydrolysate complexes in PBS (10 mM, pH 7) before thermal treatment.

FIG. 56 is a graph depicting the growth of Escherichia coli, as indicated by OD625 nm, at 37 °C in the presence of pure lactoferrin, pure whey protein hydrolysate, or lactoferrin/whey protein hydrolysate complexes in PBS (10 mM, pH 7) before thermal treatment.

FIG. 57 is a graph depicting the growth of Staphylococcus aureus, as indicated by OD625 nm, at 37 °C in the presence of pure lactoferrin, pure whey protein hydrolysate, or lactoferrin/whey protein hydrolysate complexes in PBS (10 mM, pH 7) after thermal treatment at 75 °C for 2 minutes.

FIG. 58 is a graph depicting the growth of Escherichia coli, as indicated by OD625 nm, at 37 °C in the presence of pure lactoferrin, pure whey protein hydrolysate, or lactoferrin/whey protein hydrolysate complexes in PBS (10 mM, pH 7) after thermal treatment at 75 °C for 2 minutes.

FIG. 59 is a graph depicting the growth of Staphylococcus aureus, as indicated by OD625 nm, at 37 °C in the presence of pure lactoferrin, pure whey protein hydrolysate, or lactoferrin/whey protein hydrolysate complexes in PBS (10 mM, pH 7) after thermal treatment at 90 °C for 2 minutes.

FIG. 60 is a graph depicting the growth of Escherichia coli, as indicated by OD625 nm, at 37 °C in the presence of pure lactoferrin, pure whey protein hydrolysate, or lactoferrin/whey protein hydrolysate complexes in PBS (10 mM, pH 7) after thermal treatment at 90 °C for 2 minutes.

FIG. 61 shows graphs demonstrating the effect of pH and mixing ratio on the turbidity, zeta-potential, and mean particle size of LF-WPH mixtures according to some embodiments of the present invention.

FIG. 62 shows graphs demonstrating the effect of pH and mixing ratio on the turbidity, zeta-potential, and mean particle size of LF-WPH (BIOZATE® 9) mixtures according to some embodiments of the present invention.

### DETAILED DESCRIPTION OF EXAMPLE EMBODIMENTS

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B, and C, it is specifically intended that any of A, B, or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 8%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

A “portion” or “fragment” of a nucleotide sequence or polypeptide (including a domain) will be understood to mean a nucleotide sequence or polypeptide of reduced length (e.g., reduced by 1, 2, 3, 4, 5, 6, 7, 8, 9, 10, 11, 12, 13, 14, 15, 16, 17, 18, 19, 20 or more residue(s) (e.g., nucleotide(s) or peptide(s)) relative to a reference nucleotide sequence or polypeptide, respectively, and comprising, consisting essentially of and/or consisting of a nucleotide sequence or polypeptide of contiguous residues, respectively, identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleotide sequence or polypeptide.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

As used herein, the phrase “substantially identical,” or “substantial identity” in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 9100, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, or more nucleotides in length, and any range therein, up to the full length of the sequence. In some embodiments, the nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40 nucleotides). In some embodiments, a substantially identical nucleotide or protein sequence performs substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Provided according to embodiments of the present invention are complexes comprising a lactoferrin and a whey protein hydrolysate. A complex of the present invention may be a binary complex in that the complex comprises two different components, such as a lactoferrin and a whey protein hydrolysate that are each different from each other (e.g., different in chemical structure). In some embodiments, a complex of the present invention comprises two or more (e.g., 2, 3, 4, or more) different components, such as a lactoferrin and a whey protein hydrolysate, that are each different from each other (e.g., different in chemical structure). In a complex of the present invention, the lactoferrin and the whey protein hydrolysate may be associated with one another via electrostatic interactions.

A complex of the present invention may comprise one or more lactoferrin molecule(s) (e.g., proteins) and one or more whey protein hydrolysate molecules(s) (e.g., proteins and/or peptides), which may be associated with one another via electrostatic interactions. A complex of the present invention may have a zeta potential of about −10, −9, −8, −7, −6, −5, −4, −3, −2, −1, or 0 mV to about +1, +2, +3, +4, +5, +6, +7, +8, +9, +10, +11, +12, +13, +14, or +15 mV, optionally when present in a composition (e.g., water and/or a buffer) having a pH of about 5.8 or 6 to about 6.2 or 6.5. In some embodiments, a complex of the present invention may have a zeta potential of about −10, −9, −8, −7, −6, −5, −4, −3, −2, −1, 0, +1, +2, +3, +4, +5, +6, +7, +8, +9, +10, +11, +12, +13, +14, or +15 mV, optionally when present in a composition (e.g., water and/or a buffer) having a pH of about 5.8 or 6 to about 6.2 or 6.5. In some embodiments, a complex of the present invention may have a zeta potential of about 0 mV, optionally when present in a composition (e.g., water and/or a buffer) having a pH of about 5.8 or 6 to about 6.2 or 6.5.

A complex of the present invention may comprise a lactoferrin and a whey protein hydrolysate in an amount of about 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% w/w of the complex. In some embodiments, a complex of the present invention may comprise a whey protein hydrolysate in an amount of about 1%, 5%, 10%, 15%, 20%, 25%, 30%, 35%, 40%, 45%, 50%, 55%, 60%, 65%, 70%, or 75% w/w of the complex. In some embodiments, a complex of the present invention may comprise a whey protein hydrolysate in an amount of about 1%, 5%, 10%, 15%, 20%, 25%, 30%, 35%, 40%, or 45% to about 50%, 55%, 60%, 65%, 70%, or 75% w/w of the complex. In some embodiments, a complex of the present invention may comprise a lactoferrin in an amount of about 1%, 5%, 10%, 15%, 20%, 25%, 30%, 35%, 40%, or 45% to about 50%, 55%, 60%, 65%, 70%, 75%, 80%, 85%, or 90% w/w of the complex. In some embodiments, a complex of the present invention may comprise a lactoferrin in an amount of about 40%, 45%, 50%, 55%, 60%, or 65% to about 70%, 75%, 80%, 85%, or 90% w/w of the complex. In some embodiments, a complex of the present invention may comprise a lactoferrin in an amount of about 1%, 5%, 10%, 15%, 20%, 25%, 30%, 35%, 40%, 45%, 50%, 55%, 60%, 65%, 70%, 75%, 80%, 85%, or 90% w/w of the complex.

In some embodiments, a complex of the present invention may comprise a lactoferrin in an amount of about 25%, 30%, 35%, 40%, 45% or about 50%, 55%, 60%, 65%, 70%, 75%, 80%, 85%, or 90% w/w of the complex and a whey protein hydrolysate in an amount of about 25%, 30%, 35%, or 40% to about 45%, 50%, 55%, 60%, 65%, 70%, or 75% w/w of the complex. In some embodiments, a complex of the present invention may comprises a lactoferrin in an amount of about 50% or 65% to about 75% w/w and a whey protein hydrolysate in an amount of about 25% to about 50% w/w. In some embodiments, a complex of the present invention may comprises a lactoferrin in an amount of about 50% and a whey protein hydrolysate in an amount of about 50% w/w.

One or more (e.g., 1, 2, 3, 4, 5, 10, 15, 20, 30, 40, or 50, or more) lactoferrin molecule(s) may be present in a complex of the present invention. A lactoferrin present in a complex of the present invention and/or used to prepare a complex of the present invention may have a net positive charge, optionally at a pH of about 1, 2, 3, 4, 5, 6, 7, or 8 or a pH of less than 7 or 8. In some embodiments, a lactoferrin present in a complex of the present invention and/or used to prepare a complex of the present invention, optionally when present in a composition (e.g., water and/or a buffer) having a pH of about 4, 4.5, or 5 to about 6, 6.5, or 7, may have a zeta potential of greater than about +1, +2, +3, +4, +5, +6, +7, +8, +9, or +10 mV to about +11, +12, +13, +14, +15, +16, +17, +18, +19, or +20 mV.

A lactoferrin present in a complex of the present invention and/or used to prepare a complex of the present invention may have a globular structure. In some embodiments, a lactoferrin present in a complex of the present invention and/or used to prepare a complex of the present invention may have an elongated structure made up of two globular lobes. In some embodiments, a lactoferrin present in a complex of the present invention and/or used to prepare a complex of the present invention has a radius of gyration (R_{g}) of about 4, 5, 6, 7, or 8 nm, optionally as measured by Small Angle X-Ray Scattering (SAXS). In some embodiments, the lactoferrin present in a complex of the present invention and/or used to prepare a complex of the present invention has a radius of gyration (R_{g}) of about 5.7 nm. In some embodiments, a lactoferrin present in a complex of the present invention and/or used to prepare a complex of the present invention has an intermolecular distance (Dₘₐₓ) of about 19, 20, 21, 22, 23, 24, or 25 nm, optionally as measured by Small Angle X-Ray Scattering (SAXS). In some embodiments, the lactoferrin present in a complex of the present invention and/or used to prepare a complex of the present invention has an intermolecular distance (Dₘₐₓ) of about 21.2 nm. In some embodiments, a lactoferrin present in a complex of the present invention and/or used to prepare a complex of the present invention is soluble in water at a pH of 8 or less and/or at a pH of greater than 9, such as at a pH of about 3, 3.5, 4, 4.5, 5, 5.5, 6, 6.5, 7, 7.5, 8, 9, 9.5, or 10. In some embodiments, a lactoferrin present in a complex of the present invention and/or used to prepare a complex of the present invention has a pI of in a pH range of about 7 to about 9, optionally at a pH of about 8. In some embodiments, a lactoferrin present in a complex of the present invention and/or used to prepare a complex of the present invention is a dairy protein. A “dairy protein” as used herein refers to a protein that is found naturally in a dairy product and/or milk and/or that is derived from such a naturally occurring protein to have an amino acid sequence having at least 70% sequence identity to the naturally occurring protein's amino acid sequence. For example, in some embodiments, a dairy protein is naturally found in a milk (e.g., an animal milk) and/or the protein is isolated from a milk, or the protein is synthetically prepared to have an amino acid sequence having at least 70% sequence identity to the naturally occurring protein's amino acid sequence. A lactoferrin present in and/or used to prepare a complex of the present invention may be obtained from and/or derived from an animal such as a mammal (e.g., a bovine, goat, sheep, or human).

Exemplary lactoferrins that may be present in a complex of the present invention and/or used to prepare a complex of the present invention include, but are not limited to, bovine lactoferrin or human lactoferrin. In some embodiments, a lactoferrin present in a complex of the present invention has an amino acid sequence having about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:1 and/or SEQ ID NO:2. In some embodiments, a lactoferrin present in a complex of the present invention has an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:1 and/or SEQ ID NO:2. In some embodiments, a lactoferrin present in a complex of the present invention has an amino acid sequence having about 100% sequence identity to SEQ ID NO:1 and/or SEQ ID NO:2.

One or more (e.g., 1, 2, 3, 4, 5, 10, 15, 20, 30, 40, or 50, or more) whey protein hydrolysate(s) may be present in a complex of the present invention. In some embodiments, a complex of the present invention comprises two or more whey protein hydrolysates that may be the same or different from each other. In some embodiments, a complex of the present invention comprises one or more whey protein hydrolysate molecule(s) (e.g., individual whey protein hydrolysate compounds) that are the same.

A whey protein hydrolysate present in a complex of the present invention and/or used to prepare a complex of the present invention may have a net negative charge, optionally at a pH of about 2, 3, 4, 5, 5.5, 6, 6.5, 7, 7.5, or 8 and/or at a pH of less than 7 or 8. In some embodiments, a whey protein hydrolysate present in a complex of the present invention and/or used to prepare a complex of the present invention, optionally when present in a composition (e.g., water and/or a buffer) having a pH of about 5, 5.5, or 5.8 to about 6.2 or 6.5, may have a zeta potential of about −20 mV to about −1 mV, such as a zeta potential of about −20, −19, −18, −17, −16, −15, −14, −13, −12, −11, −10, −9, −8, −7, −6, −5, −4, −3, −2, or −1. A whey protein hydrolysate present in a complex of the present invention and/or used to prepare a complex of the present invention may have a pI at a pH of about 3, 3.5, or 4 to about 4.5 or 5. In some embodiments, a whey protein hydrolysate present in a complex of the present invention and/or used to prepare a complex of the present invention may have a pI at a pH of about 4.5. In some embodiments, a whey protein hydrolysate present in a complex of the present invention and/or used to prepare a complex of the present invention may be soluble in water at a pH of about 8 or less, such as a pH of about 7.5, 7, 6.5, 6, 5.5, 5, 4.5, 4, 3.5, or 3.

In some embodiments, a whey protein hydrolysate present in a complex of the present invention and/or used to prepare a complex of the present invention has a radius of gyration (R_{g}) of about 1, 2, 3, 4, or 5 nm, optionally as measured by Small Angle X-Ray Scattering (SAXS). In some embodiments, the whey protein hydrolysate present in a complex of the present invention and/or used to prepare a complex of the present invention has a radius of gyration (R_{g}) of about 1.6 nm. In some embodiments, a whey protein hydrolysate present in a complex of the present invention and/or used to prepare a complex of the present invention has an intermolecular distance (Dₘₐₓ) of about 3, 4, 5, 6, or 7 nm, optionally as measured by Small Angle X-Ray Scattering (SAXS). In some embodiments, the whey protein hydrolysate present in a complex of the present invention and/or used to prepare a complex of the present invention has an intermolecular distance (Dₘₐₓ) of about 5.2 nm.

A “whey protein hydrolysate” as used herein refers to a whey protein (e.g., a whey protein concentrate and/or a whey protein isolate) that has been hydrolyzed by an enzyme and the hydrolysis rate (i.e., degree of hydrolysis) refers to the amount of hydrolysis. The degree of hydrolysis (DF) of a whey protein hydrolysate can be measured by methods known in the art such as, but not limited to, by measuring the percent ratio of the number of peptide bonds broken to the total numbers of bonds per unit weight of the whey protein and/or by measuring the ratio of released alpha-NH₂ groups to the total alpha-NH₂ groups in the initial protein substrate (e.g., the whey protein). As one of skill in the art recognizes, whey protein hydrolysate is different than whey protein concentrate and/or isolate. In some embodiments, a whey protein isolate and/or a whey protein concentrate is used to prepare a whey protein hydrolysate. A protease and/or a peptidase that can hydrolyze a whey protein and/or peptide thereof may be used to prepare a whey protein hydrolysate. In some embodiments, an enzyme used to prepare a whey protein hydrolysate is a trypsin, pepsin, chymotrypsin, fungal protease, papain, and/or a commercial protease mixture such as Protex 6L protease. A whey protein hydrolysate present in a complex of the present invention and/or used to prepare a complex of the present invention may have a hydrolysis rate of about 8%, 8.5%, 9%, 9.5%, 10%, or 10.5% to about 11%, 11.5%, 12%, 12.5%, or 13%. In some embodiments, a whey protein hydrolysate present in a complex of the present invention and/or used to prepare a complex of the present invention may have a hydrolysis rate of about 8%, 8.5%, 9%, 9.5%, 10%, 10.5%, 11%, 11.5%, 12%, 12.5%, or 13%. In some embodiments, a whey protein hydrolysate present in a complex of the present invention and/or used to prepare a complex of the present invention may have a hydrolysis rate of about 9% to about 11%. In some embodiments, a whey protein hydrolysate present in a complex of the present invention and/or used to prepare a complex of the present invention may have a hydrolysis rate of about 10% or about 11%. In some embodiments, a whey protein hydrolysate may be a commercially available whey protein hydrolysate such as, but not limited to, BIOZATE® 9 commercially available from Agropur.

A whey protein hydrolysate present in a complex of the present invention and/or used to prepare a complex of the present invention may comprise a mixture of peptides and/or amino acids. In some embodiments, a whey protein hydrolysate (WPH) present in a complex of the present invention and/or used to prepare a complex of the present invention may have an amino acid profile in which alanine is present in an amount of about 4 g to about 6 g per 100 g of the WPH, arginine is present in an amount of about 1 g to about 3.5 g per 100 g of the WPH, aspartic acid and/or arginine is present in an amount of about 10 g to about 12.5 g per 100 g of the WPH, cysteine is present in an amount of about 1 g to about 4 g per 100 g of the WPH, glutamic acid and/or glutamine is present in an amount of about 14 g to about 18 g per 100 g of the WPH, glycine is present in an amount of about 0.5 g to about 3 g per 100 g of the WPH, histidine is present in an amount of about 1 g to about 3 g per 100 g of the WPH, isoleucine is present in an amount of about 4 g to about 7 g per 100 g of the WPH, leucine is present in an amount of about 10 g to about 14 g per 100 g of the WPH, lysine is present in an amount of about 8 g to about 12 g per 100 g of the WPH, methionine is present in an amount of about 1 g to about 4 g per 100 g of the WPH, phenylalanine is present in an amount of about 1 g to about 5 g per 100 g of the WPH, proline is present in an amount of about 3 g to about 6 g per 100 g of the WPH, serine is present in an amount of about 1 g to about 5 g per 100 g of the WPH, threonine is present in an amount of about 3 g to about 6 g per 100 g of the WPH, tryptophan is present in an amount of about 1, 2, or 3 g to about 4, 5, or 6 g per 100 g of the WPH, tyrosine is present in an amount of about 1 g to about 5 g per 100 g WPH, and/or valine is present in an amount of about 4 g to about 7 g per 100 g WPH. In some embodiments, a whey protein hydrolysate present in a complex of the present invention and/or used to prepare a complex of the present invention comprises tryptophan in an amount of about 1, 2, 3, 4, 5, 6, 7, 8, 9, 10, 11, 12, 13, 14, 15, 61, 17, 18, 19, or 20 grams per 100 g of the whey protein hydrolysate.

In some embodiments, a whey protein hydrolysate of the present invention may comprise a mixture of peptides and/or amino acids and about 75%, 80%, 85%, or more of the mixture by weight has a molecular weight (e.g., an average molecular weight) of about 10, 11, 12, 13, 14, 15, 16, 17, 18, 19, or 20 kilodaltons (kDa) or less. In some embodiments, about 60%, 65%, or 70% by weight of a whey protein hydrolysate has a molecular weight of about 5, 4, 3, 2, or 1 kDa or less. In some embodiments, about 40%, 45%, or 50% by weight of a whey protein hydrolysate has a molecular weight of about 2, 2.5, or 1 kDa or less. Molecular weight of a protein and/or whey protein hydrolysate may be determined using methods known in the art such as chromatography methods (e.g., high-performance liquid chromatography). In some embodiments, a whey protein hydrolysate present in a complex of the present invention and/or used to prepare a complex of the present invention may have a molecular weight profile, wherein less than 5% of the whey protein hydrolysate has a molecular weight of greater than 20 kDa, less than 10% of the whey protein hydrolysate has a molecular weight in a range of 10 kDa to 20 kDa, about 5% to about 15% has a molecular weight in a range of 5 kDa to 10 k kDa, about 20% to about 30% of the whey protein hydrolysate has a molecular weight in a range of 2 kDa to 5 kDa, about 25% to about 35% of the whey protein hydrolysate has a molecular weight in a range of 1 kDa to 2 kDa, about 15% to about 25% of the whey protein hydrolysate has a molecular weight in a range of 500 Da to 1 kDa, and/or about 5% to about 15% of the whey protein hydrolysate has a molecular weight 500 Da or less, optionally as determined by high-performance liquid chromatography. In some embodiments, a whey protein hydrolysate present in a complex of the present invention and/or used to prepare a complex of the present invention may have a molecular weight profile, wherein more than 85% of the whey protein hydrolysate has a molecular weight of lower than 10 kDa, more than 70% of the whey protein hydrolysate has a molecular weight lower than 5 kDa, and more than 50% of the whey protein hydrolysate has a molecular weight lower than 2 kDa, optionally as determined by high-performance liquid chromatography.

In some embodiments, a whey protein hydrolysate present in a complex of the present invention and/or used to prepare a complex of the present invention has a particle size (e.g., diameter) in at least one dimension in a range of about 1, 5, 10, 20, 30, 40, 50, 60, 70, 80, 90, or 100 nm to about 1, 1.1, 1.2, 1.3, 1.4, 1.5, 1.6, 1.7, 1.8, 1.9, or 2 microns. In some embodiments, a whey protein hydrolysate present in a complex of the present invention and/or used to prepare a complex of the present invention may have a particle size (e.g., diameter) in at least one dimension of about 300, 250, 200, 150, or 100 nm, or less.

In some embodiments, a lactoferrin and/or a whey protein hydrolysate present in a complex of the present invention and/or used to prepare a complex of the present invention is/are each a food-grade component. A “food-grade component” as used herein refers to a component (e.g., compound, ingredient, lactoferrin, whey protein hydrolysate, etc.) that is safe for consumption by an animal (e.g., a human) and/or intended to be ingested by an animal (e.g., a human). In some embodiments, a lactoferrin and a whey protein hydrolysate of the present invention are each different food-grade components that are present in a complex of the present invention. In some embodiments, a lactoferrin and a whey protein hydrolysate in a complex of the present invention are each obtained and/or derived from a natural product (e.g., a food, plant, animal by-product (e.g., milk), etc.).

In some embodiments, a complex of the present invention is an interpolymeric complex. An “interpolymeric complex” as used herein refers to a co-precipitate or aggregate comprising a lactoferrin and a whey protein hydrolysate that is formed via electrostatic interactions. In some embodiments, a complex of the present invention is an amorphous interpolymeric complex structure. In some embodiments, a whey protein hydrolysate encapsulates and/or surrounds (e.g., partially or entirely) a lactoferrin in a complex of the present invention. In some embodiments, a complex of the present invention has a loose matrix structure. In some embodiments, a complex of the present invention has a rod-like structure. In some embodiments, a complex of the present invention has a cube-like structure. In some embodiments, each complex of a plurality of complexes of the present invention, when present in a composition (e.g., a liquid such as water, a buffer, a milk (e.g., skim milk), and/or an acid whey beverage), have the same or a different structure selected from a loose matrix structure, a rod-like structure, and/or a cube-like structure. In some embodiments, a complex of the present invention is devoid of a cationic and/or an anionic biopolymer that is not a lactoferrin or a whey protein hydrolysate. In some embodiments, a complex of the present invention is an interpolymeric complex that is formed from a cluster of two or more smaller complexes that include a lactoferrin and a whey protein hydrolysate, optionally wherein one or both of the lactoferrin and the whey protein hydrolysate molecules are nanoparticles and/or have at least one dimension less than 200 nm or 100 nm. In some embodiments, a complex of the present invention is an interpolymeric complex that is formed from a cluster of two or more smaller complexes that include a lactoferrin and a whey protein hydrolysate, wherein one or both of the lactoferrin and the whey protein hydrolysate molecules are nanoparticles and/or have a mean particle size of about 100 nm or less (e.g., less than 100 nm).

A complex of the present invention may be a particle. In some embodiments, the complex is a nanoparticle. In some embodiments, the complex is a microparticle. A complex of the present invention may have a size (e.g., a diameter) in at least one dimension of about 300, 350, 400, 450, 500, 550, 600, 650, 700, 750, 800, 850, 900, 1000, 2000, 3000, 4000, 5000, or 6000 nm or more, optionally as measured using microscopy (e.g., optical microscopy, confocal microscopy, scanning electron microscopy (SEM) and/or transmission electron microscopy (TEM)) and/or dynamic light scattering (DLS). In some embodiments, the particle has a size (e.g., a diameter) in at least one dimension of about 25, 50, 75, 100, 125, 150, 200, 250, 300, 350, 400, 450, 500, 550, 600, 650, 700, 750, 800, 850, 900, 1000, or 1500 to about 2000, 2500, 3000, 3500, or 4000 nm or more. In some embodiments, a complex of the present invention in a liquid composition (e.g., an aqueous composition) has an average size (e.g., diameter) in at least one dimension of about 300, 400, 500, 600, 700, 800, 900, 1000, or 1500 nm to about 2000, 2500, 3000, 3500, or 4000 nm or more. In some embodiments, a lactoferrin and a whey protein hydrolysate in a complex of the present invention are nanoparticles and/or have at least one dimension less than 200 nm or 100 nm. In some embodiments, a lactoferrin and a whey protein hydrolysate in a complex of the present invention have at least one dimension of about 10, 20, 30, 40, 50, 60, 70, 80, 90, or 100 nm or any range therein. In some embodiments, a lactoferrin and/or a whey protein hydrolysate in a complex of the present invention have a mean particle size of about 100 nm or less (e.g., less than 100, 90, 80, 70, 60, 50, or 40 nm). In some embodiments, a lactoferrin and/or a whey protein hydrolysate in a complex of the present invention have a mean particle size about 10, 20, 30, 40, 50, 60, 70, 80, 90, or 100 nm or any range therein.

In some embodiments, a plurality of complexes of the present invention, when present in a composition (e.g., a liquid such as water, a buffer, a milk (e.g., skim milk), and/or an acid whey beverage), have a particle size distribution of about 100, 500, 1,000, 2,000, 3,000, 4,000, or 5,000 nm to about 6,000, 7,000, 8,000, 9,000, or 10,000 nm, optionally as measured using microscopy (e.g., optical microscopy, confocal microscopy, scanning electron microscopy (SEM), and/or transmission electron microscopy (TEM)) and/or dynamic light scattering (DLS). In some embodiments, a plurality of complexes of the present invention, when present in a composition (e.g., a liquid such as water, a buffer, a milk (e.g., skim milk), and/or an acid whey beverage), have a mean particle size (e.g., a mean particle diameter) of about 500, 1,000, 1,200, or 1,300 nm to about 1,400, 1,500, 2000, 3000, or 4000 nm, optionally as measured using microscopy (e.g., optical microscopy, confocal microscopy, scanning electron microscopy (SEM), and/or transmission electron microscopy (TEM)) and/or dynamic light scattering (DLS).

In some embodiments, a complex of the present invention has a radius of gyration (R_{g}) of about 10, 11, 12, 13, 14, or 15 nm to about 16, 17, 18, 19, or 20 nm, optionally as measured by Small Angle X-Ray Scattering (SAXS). In some embodiments, a complex of the present invention has a radius of gyration (R_{g}) of about 10, 11, 12, 13, 14, 15, 16, 17, 18, 19, or 20 nm, optionally as measured by Small Angle X-Ray Scattering (SAXS). In some embodiments, a complex of the present invention has a radius of gyration (R_{g}) of about 13.2 nm, optionally as measured by Small Angle X-Ray Scattering (SAXS). In some embodiments, a complex of the present invention has a radius of gyration (R_{g}) of about 18.8 nm, optionally as measured by Small Angle X-Ray Scattering (SAXS). In some embodiments, a complex of the present invention, when present in a composition of the present invention, has an intermolecular distance (Dₘₐₓ) of about 30, 35, 40, or 45 nm to about 50, 55, 60, 65, or 70 nm, optionally as measured by Small Angle X-Ray Scattering (SAXS). In some embodiments, a complex of the present invention, when present in a composition of the present invention, has an intermolecular distance (Dₘₐₓ) of about 30, 35, 40, 45, 50, 55, 60, 65, or 70 nm, optionally as measured by Small Angle X-Ray Scattering (SAXS). In some embodiments, a complex of the present invention, when present in a composition of the present invention having a ratio of lactoferrin:whey protein hydrolysate of 2:1, has an intermolecular distance (Dₘₐₓ) of about 41.5 nm, optionally as measured by Small Angle X-Ray Scattering (SAXS). In some embodiments, a complex of the present invention, when present in a composition of the present invention having a ratio of lactoferrin:whey protein hydrolysate of 1:1, has an intermolecular distance (Dₘₐₓ) of about 60.9 nm, optionally as measured by Small Angle X-Ray Scattering (SAXS).

A complex of the present invention may be dried, optionally by freeze-drying and/or spraying-drying a composition (e.g., an aqueous composition) comprising the complex. In some embodiments, a dried complex of the present invention comprises water in an amount of about 0% to about 5% by weight of the dried complex. In some embodiments, a dried complex of the present invention is devoid of water. In some embodiments, a complex of the present invention is crosslinked, optionally crosslinked using a crosslinker such as, but not limited to, transglutaminase, glyceraldehyde, dialdehydic pectin, and/or genipin.

In some embodiments, a complex of the present invention comprises an active ingredient. The active ingredient may be present within (e.g., entrapped and/or encapsulated within) the complex. In some embodiments, the active ingredient may be bound (e.g., covalently and/or noncovalently) to a lactoferrin and/or whey protein hydrolysate present in the complex. Exemplary active ingredients include, but are not limited to, amino acids (e.g., tryptophan, leucine, phenylalanine, cysteine, and/or tyrosine), vitamin E, iron, vitamin A, vitamin D, and any combination thereof.

A complex of the present invention may have improved (e.g., increased) storage, stability (e.g., thermal stability), activity, and/or function for a component (e.g., a lactoferrin and/or a whey protein hydrolysate) present in the complex compared to the storage, stability, activity, and/or function of the component alone (i.e., the component not present in a complex of the present invention). In some embodiments, a complex of the present invention provides increased stability for a component (e.g., a lactoferrin) present in the complex compared to the stability of the component alone.

In some embodiments, a lactoferrin present in a complex of the present invention has increased stability (e.g., reduced degradation of the lactoferrin) after exposure to a temperature in a range of about 70 °C or 75 °C to about 80 °C, 85 °C, 90 °C, 95 °C, or 100 °C for about 1, 2, 3, 4, 5, or 10 minute(s) to about 15, 20, 30, 40, 50, or 60 minutes compared to the stability of the lactoferrin alone (i.e., the lactoferrin not present in a complex of the present invention) after exposure to the same conditions (e.g., the same temperature for the same period of time). In some embodiments, a lactoferrin present in a complex of the present invention has increased stability (e.g., reduced degradation of the lactoferrin) after exposure to a temperature of about 100 °C, 105 °C, 110 °C, 115 °C, or 120 °C to about 125 °C, 130 °C, 135 °C, 140 °C, or 145 °C for about 2, 5, 10, 20, or 30 seconds to about 40, 50, or 60 seconds as compared to the stability of the lactoferrin alone (i.e., the lactoferrin not present in a complex of the present invention) after exposure to the same conditions (e.g., the same temperature for the same period of time). In some embodiments, a lactoferrin present in a complex of the present invention has increased stability (e.g., reduced degradation of the lactoferrin) after exposure to a high-temperature short time (HTST) treatment in an oil bath condition at about 145 °C for about 2 seconds to about 60 seconds as compared to the stability of the lactoferrin alone (i.e., the lactoferrin not present in a complex of the present invention) after exposure to the same conditions (e.g., the same temperature for the same period of time). In some embodiments, the complex may be present in a composition (e.g., a liquid such as water, a buffer, a milk (e.g., skim milk), and/or an acid whey beverage) and exposed to the temperature and/or HTST treatment. In some embodiments, increased stability for the lactoferrin is determined and/or demonstrated by reduced degradation of the lactoferrin in the complex compared to the degradation of the lactoferrin alone.

In some embodiments, after exposure to a temperature in a range of about 70 °C or 75 °C to about 80 °C, 85 °C, 90 °C, 95 °C, or 100 °C for about 1, 2, 3, 4, 5, or 10 minute(s) to about 15, 20, 30, 40, 50, or 60 minutes, a lactoferrin present in a complex of the present invention is degraded by less than 50% such as about 45%, 40%, 35%, 30%, 25%, 20%, 15%, 10%, 5%, 1%, or less, optionally as measured by chromatography (e.g., high-performance liquid chromatography). In some embodiments, after exposure to a temperature in a range of about 70 °C or 75 °C to about 80 °C, 85 °C, 90 °C, 95 °C, or 100 °C for about 1, 2, 3, 4, 5, or 10 minute(s) to about 15, 20, 30, 40, 50, or 60 minutes, a lactoferrin present in a complex of the present invention is degraded by less than 40% such as about 35%, 30%, 25%, 20%, 15%, 10%, 5%, 1%, or less, optionally as measured by high-performance liquid chromatography. In some embodiments, after exposure to a temperature in a range of about 100 °C, 105 °C, 110 °C, 115 °C, or 120 °C to about 125 °C, 130 °C, 135 °C, 140 °C, or 145 °C for about 2, 5, 10, 20, or 30 seconds to about 40, 50, or 60 seconds, a lactoferrin present in a complex of the present invention is degraded by less than about 30% such as about 25%, 20%, 15%, 10%, 5%, 1%, or less, optionally as measured by chromatography (e.g., high-performance liquid chromatography). In some embodiments, after exposure to a high-temperature short time (HTST) treatment in an oil bath at about 145 °C for about 2 seconds to about 60 seconds, a lactoferrin present in a complex of the present invention is degraded by less than about 30% such as about 25%, 20%, 15%, 10%, 5%, 1%, or less, optionally as measured by chromatography (e.g., high-performance liquid chromatography). In some embodiments, after exposure to a temperature in a range of about 100 °C, 105 °C, 110 °C, 115 °C, or 120 °C to about 125 °C, 130 °C, 135 °C, 140 °C, or 145 °C for about 2, 5, 10, 20, or 30 seconds to about 40, 50, or 60 seconds and/or a HTST treatment in an oil bath condition at about 145 °C for about 2 seconds to about 60 seconds, a lactoferrin present in a complex of the present invention is degraded by less than about 20% such as about 15%, 10%, 5%, 1%, or less, optionally as measured by chromatography (e.g., high-performance liquid chromatography).

In some embodiments, the antimicrobial capacity and/or activity (e.g., the antibacterial activity on Gram-positive and/or Gram-negative bacteria) of a lactoferrin present in a complex of the present invention, after exposure to a temperature in a range of about 70 °C or 75 °C to about 80 °C, 85 °C, or 90 °C for about 30 seconds to about 2 minutes, is retained and/or improved (e.g., increased) as compared to the antimicrobial capacity and/or activity of the lactoferrin alone, optionally after exposure to the same conditions (e.g., same temperature and time). In some embodiments, the antimicrobial capacity and/or activity (e.g., the antibacterial activity on Gram-positive and/or Gram-negative bacteria) of a lactoferrin present in a complex of the present invention, after exposure to a temperature in a range of about 70 °C or 75 °C to about 80 °C, 85 °C, or 90 °C for about 30 seconds to about 2 minutes, is retained and/or increased as compared to the antimicrobial capacity and/or activity of the lactoferrin alone, optionally after exposure to the same conditions (e.g., same temperature and time).

In some embodiments, upon exposure to a temperature in a range of about 70 °C or 75 °C to about 80 °C, 85 °C, or 90 °C for about 30 seconds to about 2 minutes or to a temperature in a range of about 100 °C, 105 °C, 110 °C, 115 °C, or 120 °C to about 125 °C, 130 °C, 135 °C, 140 °C, or 145 °C for about 2, 5, 10, 20, or 30 seconds to about 40, 50, or 60 seconds, the amount of a component (e.g., a lactoferrin and/or a whey protein hydrolysate) present in a complex of the present invention that is in a composition (e.g., a liquid such as water, a buffer, a milk (e.g., skim milk), and/or an acid whey beverage) remains within about +30% of the amount of the component present in the complex prior to the exposure (e.g., the amount of the component present in the complex at initial formation of the complex and/or immediately prior to the exposure), optionally as measured by chromatography (e.g., high-performance liquid chromatography). For example, the amount of a component (e.g., a lactoferrin and/or a whey protein hydrolysate) present in a complex of the present invention following exposure of the complex to a temperature for a period of time may be about 70% or more (e.g., about 75%, 80%, 85%, 90%, 95%, or 100%) of the amount of the same component present in the complex prior to the exposure to the temperature for the period of time. In some embodiments, an activity (e.g., bioactivity and/or antibacterial activity) and/or function of a component (e.g., a lactoferrin and/or a whey protein hydrolysate) present in a complex of the present invention that is in a composition (e.g., a liquid such as water, a buffer, a milk (e.g., skim milk), and/or an acid whey beverage), upon exposure of the composition to a temperature in a range of about 70 °C or 75 °C to about 80 °C, 85 °C, or 90 °C for about 30 seconds to about 2 minutes or to a temperature in a range of about 100 °C, 105 °C, 110 °C, 115 °C, or 120 °C to about 125 °C, 130 °C, 135 °C, 140 °C, or 145 °C for about 2, 5, 10, 20, or 30 seconds to about 40, 50, or 60 seconds, remains within about +30% of the activity (e.g., bioactivity) and/or function of the component present in the complex prior to the exposure (e.g., the activity and/or function of the component present in the complex at initial formation of the complex and/or immediately prior to the exposure). For example, following exposure of a complex of the present invention to a temperature for a period of time, an activity and/or function of a component present in the complex may be about 70% or more (e.g., about 75%, 80%, 85%, 90%, 95%, or 100%) of the activity and/or function of the same component present in the complex prior to the exposure to the temperature for the period of time. In some embodiments, a physiochemical property (e.g., turbidity and/or particle size) of a complex of the present invention that is present in in a composition (e.g., a liquid such as water, a buffer, a milk (e.g., skim milk), and/or an acid whey beverage), upon exposure to a temperature in a range of about 70 °C or 75 °C to about 80 °C, 85 °C, or 90 °C for about 30 seconds to about 2 minutes or to a temperature in a range of about 100 °C, 105 °C, 110 °C, 115 °C, or 120 °C to about 125 °C, 130 °C, 135 °C, 140 °C, or 145 °C for about 2, 5, 10, 20, or 30 seconds to about 40, 50, or 60 seconds, remains within about ±30% of its original physiochemical property of the complex prior to the exposure (e.g., the physiochemical property of the complex at initial formation of the complex and/or immediately prior to the exposure). For example, following exposure of a complex of the present invention to a temperature for a period of time, a physiochemical property of the complex may be about 70% or more (e.g., about 75%, 80%, 85%, 90%, 95%, or 100%) of the physiochemical property of the complex prior to the exposure to the temperature for the period of time. In some embodiments, the amount of a component present in a complex of the present invention, an activity and/or function of a component present in a complex of the present invention, and/or a physiochemical property of a complex of the present invention may not change much (e.g., by about 30% or less) following exposure to a temperature in a range of about 70 °C or 75 °C to about 80 °C, 85 °C, or 90 °C for about 30 seconds to about 2 minutes or to a temperature in a range of about 100 °C, 105 °C, 110 °C, 115 °C, or 120 °C to about 125 °C, 130 °C, 135 °C, 140 °C, or 145 °C for about 2, 5, 10, 20, or 30 seconds to about 40, 50, or 60 seconds compared to the amount, activity and/or function, and/or physiochemical property prior to the exposure.

In some embodiments, the antimicrobial capacity and/or activity (e.g., the antibacterial activity on Gram-positive and/or Gram-negative bacteria) of a component (e.g., a lactoferrin and/or a whey protein hydrolysate) present in a complex of the present invention that is present in a composition (e.g., a liquid such as water, a buffer, a milk (e.g., skim milk), and/or an acid whey beverage), upon exposure to a temperature in a range of about 70 °C or 75 °C to about 80 °C, 85 °C, or 90 °C for about 30 seconds to about 2 minutes or to a temperature in a range of about 100 °C, 105 °C, 110 °C, 115 °C, or 120 °C to about 125 °C, 130 °C, 135 °C, 140 °C, or 145 °C for about 2, 5, 10, 20, or 30 seconds to about 40, 50, or 60 seconds, is retained and/or within about +30% of the antimicrobial capacity and/or activity of the component prior to the exposure (e.g., the antibacterial activity of the component present in the complex at initial formation of the complex and/or immediately prior to the exposure). For example, following exposure of a complex of the present invention to a temperature for a period of time, the antimicrobial capacity and/or activity of a component present in the complex may be about 70% or more (e.g., about 75%, 80%, 85%, 90%, 95%, or 100%) of the antimicrobial capacity and/or activity of the component present in the complex prior to the exposure to the temperature for the period of time.

In some embodiments, a complex of the present invention increases the thermal stability of a component (e.g., a lactoferrin and/or a whey protein hydrolysate) present in the complex compared to the thermal stability of the component alone. For example, the presence of the component in the complex may reduce or avoid denaturation (e.g., thermal denaturation such as thermal denaturation during the preparation of a food product comprising the component) of the component compared to the amount of denaturation of the component alone (i.e., the component not present in a complex of the present invention) under the same conditions. In some embodiments, a complex of the present invention increases the thermal stability of lactoferrin present in the complex upon exposure to a temperature for a period of time (e.g., a temperature in a range of about 70 °C or 75 °C to about 80 °C, 85 °C, 90 °C, 95 °C, or 100 °C for about 1, 2, 3, 4, 5, or 10 minute(s) to about 15, 20, 30, 40, 50, or 60 minutes) compared to the thermal stability of lactoferrin alone upon exposure to the same conditions (e.g., the same temperature and time). In some embodiments, a complex of the present invention increases the stability (e.g., thermal stability) structure, activity, and/or function of a component (e.g., lactoferrin) present in the complex upon exposure to a pH in a range of about 5 or 5.5 to about 6, 6.5, or 7 compared to the stability, structure, activity, and/or function of the component alone upon exposure to the same conditions (e.g., the same pH).

Activity and/or function of a component (e.g., a lactoferrin and/or a whey protein hydrolysate) present in a complex of the present invention may be increased compared to the activity and/or function of the component alone. For example, after heating a complex (e.g., at a temperature in a range of about 70 °C or 75 °C to about 80 °C, 85 °C, 90 °C, 95 °C, or 100 °C for about 1, 2, 3, 4, 5, or 10 minute(s) to about 15, 20, 30, 40, 50, or 60 minutes), a component (e.g., lactoferrin) present in the complex may have an activity and/or function that is increased compared to the activity and/or function of the component (e.g., lactoferrin) alone after the same heating conditions. In some embodiments, after storing a complex of the present invention for a period of time (e.g., storing a dried complex in a closed container at a temperature in a range of about 20 °C to about 30 °C for about 1, 2, 3, 4, 5, or 6 months) a component (e.g., lactoferrin) present in the complex may have an activity and/or function that is increased compared to the activity and/or function of the component (e.g., lactoferrin) alone after the same storage and/or heating conditions. In some embodiments, a complex comprising a lactoferrin and a whey protein hydrolysate may provide an increased activity and/or function (e.g., increased antimicrobial activity) after heating and/or storing the complex compared to the activity and/or function of lactoferrin after the same heating and/or storage conditions. In some embodiments, after exposure of a complex of the present invention to a temperature of about 70 °C to about 80 °C, 90 °C, or 100 °C for about 1 minute to about 60 minutes, the activity of lactoferrin is increased compared to the activity of lactoferrin alone after the same exposure conditions. In some embodiments, other than a whey protein hydrolysate, a complex of the present invention is devoid of an agent configured to preserve and/or stabilize the activity, function, and/or stability (e.g., thermal stability) of lactoferrin present in the complex.

In some embodiments, a complex of the present invention may provide increased bioavailability for a component (e.g., a lactoferrin and/or a whey protein hydrolysate) present in a complex of the present invention compared to the bioavailability of the component alone. Bioavailability may be determined following administration of the complex to a subject, optionally wherein administration comprises ingestion of the complex by the subject. In some embodiments, after ingestion of the complex by a subject, the bioavailability of the lactoferrin is increased compared to the bioavailability of the lactoferrin after ingestion by a subject. In some embodiments, a component (e.g., lactoferrin) present in a complex of the present invention has increased bioavailability in the intestinal tract of a subject compared to the bioavailability of the component alone. In some embodiments, a component (e.g., lactoferrin) present in a complex of the present invention has reduced enzymatic hydrolysis (e.g., reduced enzymatic hydrolysis in the gastric phase of digestion in a subject) compared to the amount of enzymatic hydrolysis of the component alone.

According to some embodiments, a composition comprising a complex of the present invention is provided and/or an article comprising a complex of the present invention is provided. In some embodiments, the composition and/or article comprises a plurality of complexes of the present invention. In some embodiments, the complex is in the form of a particle, optionally wherein the particle is a freeze-dried, spray-dried, and/or the like particle. In some embodiments, the composition and/or article comprises a plurality of particles, optionally wherein the composition and/or article is in the form of a powder (e.g., a dry powder). In some embodiments, a composition of the present invention is an aqueous composition and a complex of the present invention may be dissolved and/or suspended in the composition.

“Free lactoferrin” as used herein refers to lactoferrin that is not associated with whey protein hydrolysate via electrostatic interactions. In some embodiments, a complex of the present invention and/or a particle comprising the complex may be prepared and/or isolated from a solution comprising less than 6%, 5%, 4%, 3%, 2%, or 1% w/v free lactoferrin. In some embodiments, a complex of the present invention and/or a particle comprising the complex may be prepared and/or isolated from a solution comprising less than 1% w/v free lactoferrin. In some embodiments, a composition of the present invention, an article of the present invention, a plurality of particles of the present invention may comprise less than 6%, 5%, 4%, 3%, 2%, or 1% by weight free lactoferrin. In some embodiments, the composition, article, and/or plurality of particles may comprise less than 1% by weight free lactoferrin.

In some embodiments, a composition and/or article of the present invention comprises a complex of the present invention and a carrier. The carrier may be a liquid such as, but not limited to, water, a buffer, milk, food product, and/or an oil. In some embodiments, a composition of the present invention is an aqueous composition and the complex is dissolved and/or suspended in the composition. In some embodiments, a composition of the present invention is a suspension, optionally wherein a complex of the present invention is suspended in the composition. In some embodiments, a complex of the present invention stabilizes a composition comprising the complex, optionally wherein the composition is an emulsion. In some embodiments, the carrier is a solid (e.g., a particulate and/or powder) and a plurality of complexes of the present invention may be present together with the solid, optionally present on, below, combined with and/or mixed with the solid. In some embodiments, the carrier is a food-grade component such as, but not limited to, a milk, a dairy beverage, an infant formula, and/or an instant beverage powder. One or more excipient(s) such as, but not limited to, gum arabic, sodium caseinate, and/or maltodextrin may be present in a composition of the present invention.

In some embodiments, a composition and/or article of the present invention is a food product, nutritional supplement, therapeutic drink, and/or cosmetic. In some embodiments, a complex of the present invention may be present in a food product. In some embodiments, the food product is a dairy product (e.g., milk, yogurt, etc.). In some embodiments, the composition is an infant formula and/or a nutritional supplement (optionally a drink).

Provided according to some embodiments of the present invention is a method for preparing a complex of the present invention. In some embodiments, the method comprises providing a composition comprising a lactoferrin and a whey protein hydrolysate at a pH in a range of about 5, 5.5, or 5.8 to about 6, 6.5, or 7; and mixing the composition, thereby providing the complex. The composition comprising the lactoferrin and the whey protein hydrolysate may be an aqueous composition that optionally includes a buffer. In some embodiments, the composition used to prepare a complex of the present invention has a pH of about 5, 5.5, 5.8, 6, 6.5, or 7. In some embodiments, the composition used to prepare a complex of the present invention comprises a salt, optionally wherein the composition comprises a salt in an amount of about 0.1, 0.5, 1, or 5 mM to about 10, 15, 20, 25, or 30 mM. In some embodiments, the composition used to prepare a complex of the present invention comprises a salt in an amount of about 0.1, 0.5, 1, 5, 10, 15, 20, 25, or 30 mM.

A composition used to prepare a complex of the present invention may comprise a lactoferrin and a whey protein hydrolysate each independently present in the composition in an amount of about 0.01%, 0.1%, 0.5%, or 1% to about 2% or 3% by weight of the composition. In some embodiments, a composition used to prepare a complex of the present invention may comprise a lactoferrin and a whey protein hydrolysate each independently present in the composition in an amount of about 0.01%, 0.1%, 0.5%, 1%, 1.5%, 2%, 2.5%, or 3% by weight of the composition. In some embodiments, a composition used to prepare a complex of the present invention comprises a lactoferrin and a whey protein hydrolysate in a weight ratio of about 10:1 to about 1:10 (lactoferrin:whey protein hydrolysate) such as in a weight ratio of about 1:1 or about 2:1 (lactoferrin:whey protein hydrolysate). In some embodiments, a composition used to prepare a complex of the present invention has a total concentration of a lactoferrin and a whey protein hydrolysate in the composition in an amount of about 5% by weight of the composition or less such as about 0.05%, 0.1%, 0.5%, 1%, 1.5%, 2%, 2.5%, 3%, 3.5%, 4%, 4.5%, or 5% by weight of the composition.

Mixing a composition used to prepare a complex of the present invention may be carried out using methods known in the art. In some embodiments, mixing a composition used to prepare a complex of the present comprises mixing the composition for about 1, 5, 10, 15, 20, 25, or 30 minutes to about 35, 40, 45, 50, 55, or 60 minutes, optionally at a temperature in a range of about 20 °C, 25 °C, or 30 °C to about 35 °C, 40 °C, 45 °C, 50 °C, 55 °C, or 60 °C.

A method of the present invention may further comprise hardening a complex of the present invention. In some embodiments, hardening the complex comprises adjusting the temperature of a composition comprising a complex of the present invention to a temperature in a range of about 5 °C to about 10 °C and exposing the composition to a temperature in a range of about 5 °C to about 10 °C for about 1 or 2 hour(s) to about 3, 4, 5, or 6 hours.

In some embodiments, a method of the present invention comprises isolating and/or obtaining a complex of the present invention from a composition. Isolating and/or obtaining a complex of the present invention from a composition may comprise centrifuging, drying, freeze-drying, filtering, and/or spray-drying the composition to thereby isolate and/or obtain the complex. In some embodiments, the isolated and/or obtained complex is a dried complex, optionally wherein the dried complex comprises water in an amount of about 0% to about 5% by weight of the dried complex. A dried complex may be in the form of a particulate and/or powder. In some embodiments, the isolated and/or obtained complex is milled, ground, and/or micronized to provide a desired size such as particles having a size (e.g., diameter) of less than about 1 mm. In some embodiments, a complex of the present invention may be crosslinked using a crosslinker such as, but not limited to, transglutaminase, glyceraldehyde, dialdehydic pectin, and/or genipin. A lactoferrin and a whey protein hydrolysate may be crosslinked in a complex of the present invention.

In some embodiments, a method of the present invention comprises combining a complex of the present invention with a carrier, optionally wherein the carrier is a liquid or a solid. In some embodiments, a complex of the present invention is added to a food-grade component and/or to a food product (e.g., a beverage or a powder formula). Combining a complex of the present invention to a carrier may comprise mixing an isolated and/or obtained complex into a carrier and/or mixing a complex of the present invention that is present in a composition (e.g., an aqueous composition) into a carrier. In some embodiments, a complex of the present invention is dispersed in a carrier optionally by mixing, stirring, homogenizing, and/or the like at a temperature in a range of about 20 °C, 25 °C, or 30 °C to about 35 °C, 40 °C, 45 °C, 50 °C, 55 °C, or 60 °C.

A method of the present invention may comprise providing a therapeutic effect and/or benefit to a subject and/or treating and/or preventing a disease, disorder, and/or condition in a subject. The method may comprise administering (e.g., orally administering) a complex of the present invention and/or a composition of the present invention to a subject, optionally wherein the administering comprises the subject ingesting the complex and/or composition.

In some embodiments, a method of the present invention comprises administering a therapeutically effective amount of a complex of the present invention and/or a composition of the present invention to a subject. As used herein, the term “therapeutically effective amount” refers to an amount of complex and/or composition of the present invention that elicits a therapeutically useful response in a subject. Those skilled in the art will appreciate that the therapeutic effects need not be complete or curative, as long as some benefit is provided to the subject.

“Treat,” “treating” or “treatment of” (and grammatical variations thereof) as used herein refer to any type of treatment that imparts a benefit to a subject and may mean that the severity of the subject's condition is reduced, at least partially improved or ameliorated and/or that some alleviation, mitigation or decrease in at least one clinical symptom associated with the subject's condition is achieved and/or there is a delay in the progression of the symptom. In some embodiments, the severity of a symptom associated with iron deficiency may be reduced in a subject compared to the severity of the symptom in the absence of a method of the present invention. In some embodiments, a complex of the present invention and/or a composition of the present invention is administered to a subject to improve iron delivery and/or adsorption in a subject and/or to treat a disease and/or a symptom thereof.

In some embodiments, a complex of the present invention and/or a composition of the present invention may be administered in a treatment effective amount. A “treatment effective” amount as used herein is an amount that is sufficient to treat (as defined herein) a subject. Those skilled in the art will appreciate that the therapeutic effects need not be complete or curative, as long as some benefit is provided to the subject. In some embodiments, a treatment effective amount may be achieved by administering a complex and/or composition of the present invention to a subject, optionally wherein the administering comprises the subject ingesting the complex and/or composition.

As used herein, the terms “prevent,” “preventing” and “prevention” (and grammatical variations thereof) refer to avoidance, reduction and/or delay of the onset of a symptom associated with a disease, disorder, or condition and/or a reduction in the severity of the onset of symptom associated with a disease, disorder, or condition relative to what would occur in the absence of a method of the present invention. The prevention can be complete, e.g., the total absence of the symptom. The prevention can also be partial, such that the occurrence of the symptom in the subject and/or the severity of onset is less than what would occur in the absence of a method of the present invention. In some embodiments, a complex of the present invention and/or a composition of the present invention is administered to a subject to prevent a disease, disorder, or condition.

In some embodiments, a complex of the present invention and/or a composition of the present invention may be administered in a prevention effective amount. A “prevention effective” amount as used herein is an amount that is sufficient to prevent (as defined herein) a symptom associated with a disease, disorder, or condition in a subject. Those skilled in the art will appreciate that the level of prevention need not be complete, as long as some benefit is provided to the subject. In some embodiments, a prevention effective amount may be achieved by administering a complex and/or composition of the present invention to a subject, optionally wherein the administering comprises the subject ingesting the complex and/or composition.

The present invention finds use in both veterinary and medical applications. Subjects suitable to be treated with a method of the present invention include, but are not limited to, mammalian subjects. Mammals of the present invention include, but are not limited to, canines, felines, bovines, caprines, equines, ovines, porcines, rodents (e.g., rats and mice), lagomorphs, primates (e.g., simians and humans), non-human primates (e.g., monkeys, baboons, chimpanzees, gorillas), and the like, and mammals in utero. Any mammalian subject in need of being treated according to the present invention is suitable. Human subjects of both genders and at any stage of development (i.e., neonate, infant, juvenile, adolescent, adult) may be treated according to the present invention. In some embodiments of the present invention, the subject is a mammal and in certain embodiments the subject is a human. Human subjects include both males and females of all ages including fetal, neonatal, infant, juvenile, adolescent, adult, and geriatric subjects as well as pregnant subjects. In particular embodiments of the present invention, the subject is a human adolescent and/or adult.

A method of the present invention may also be carried out on animal subjects, particularly mammalian subjects such as mice, rats, dogs, cats, livestock and horses for veterinary purposes, and/or for drug screening and drug development purposes.

In some embodiments, the subject is “in need of” or “in need thereof” a method of the present invention, for example, if the subject has findings typically associated with a disease, disorder, or condition, is suspected to have a disease, disorder, or condition, and/or the subject has a disease, disorder, or condition.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1

### 1. Introduction

The biological function of lactoferrin (LF) is highly related to its native protein structures and conformation. Commercial thermal processing procedures have been shown to largely denaturalize LF, modify its native structures, and impact its biological function. Since the thermal denaturation temperature of natural LF is close to the pasteurization conditions of most food products, the loss of functionality of LF during thermal processing limits its final application and bioactivity in final commercial production.

We developed LF-Whey protein hydrolysate (WPH) complexes that improve the thermal stability of LF.

### 2. Materials and Methods

### 2.1 Materials

Lactoferrin (LF) powder (natural bovine LF, Bioferrin 2000, Iron>15 mg/100 g) was provided by Glanbia Nationals, Inc, Fitchburg, WI). Whey protein hydrolysate (WPH) powder (BIOZATE 9, hydrolysis rate 10-11%) was provided by Agropur USA). Hydrochloric acid and sodium hydroxide were purchased from Fisher Scientific (Hampton, NH, USA).

### 2.2 Experimental Approach

Lactoferrin (LF) solution (1%, w/v) and whey protein hydrolysate (WPH) solution (1%, w/v) were prepared by dissolving LF powder or WPH powder in Milli-Q water using a magnetic stirrer for 1 hour and storing overnight in a −4 °C refrigerator to ensure complete dissolution.

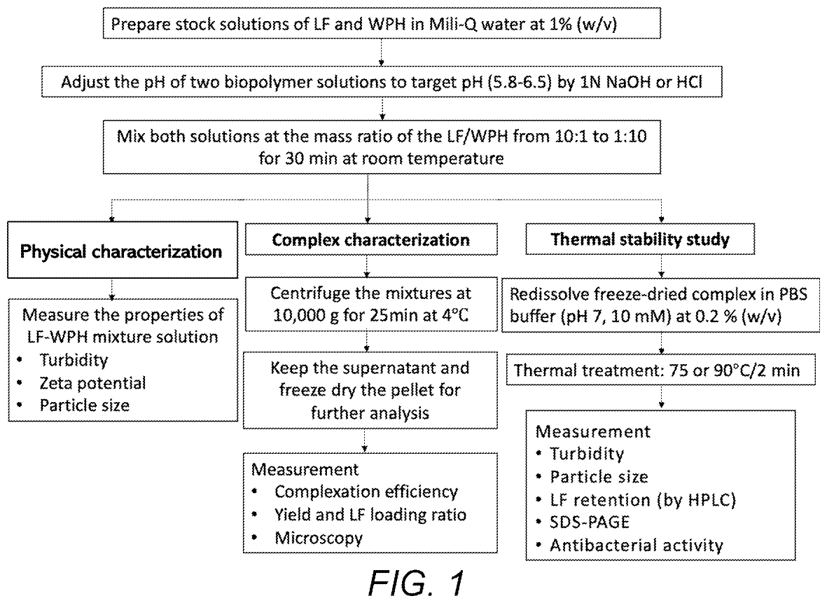

FIG. 1 shows an exemplary experimental design schematic for the formation of the LF/WPH complex. The pH of the LF solution or WPH solution was adjusted to the target pH (5.8, 6, 6.2, or 6.5) using 0.1 M NaOH or HCl. Then, the LF and WPH solutions under the same pH conditions were mixed in a certain ratio with 1% w/v total protein concentration using a magnetic stirring bar at 25 °C. After mixing for 30 minutes, the turbidity, zeta potential, and particle size of the mixing solution were measured. The LF/WPH mixing solution was then centrifuged at 10000 g for 20 minutes at 4 °C and the biopolymer-rich phase (pellet) was collected, freeze-dried, weighed, and used for the complex characterization and thermal study.

The effect of total concentrations (1, 3, 5, and 10% w/v) and mixing temperature (45 °C) and time (0-30 minutes) on complex formation were also investigated by comparing the turbidity, zeta-potential, and particle size of mixture solutions.

### 2.3 Characterization

### 2.3.1 Turbidity Measurement

The turbidity of LF/WPH mixtures was measured using a UV-Vis light spectrophotometer (UV-2600, SHIMADZU Co., Japan). The transmittance was measured at 600 nm in 1 cm path-length quartz cuvettes at room temperature. Milli-Q water was used as blank (100% transmittance). The turbidity (T) was calculated according to the following equation (Eq.1):

$$T = - ln \frac{I}{{I}_{0}} \qquad ( Eq . 1 )$$

where I is the transmittance intensity of samples and I₀ is the transmittance intensity of blank.

### 2.3.2 Particle Size Measurement

The average diameter and particle size distribution of LF/WPH mixtures were analyzed using the dynamic light scattering instrument (Zetasizer Nano-ZS, Malvern, Germany). All analyses were performed at 25 °C in a 1-cm path-length cuvette at the wavelength of 633 nm and a backscattering angle of 173°. The refractive index of dispersant was set as 1.33 and the refractive index of material was set as 1.45. Analysis was done in triplicate with at least 11 runs for each measurement.

### 2.3.3 Zeta-Potential Measurement

The zeta potential of LF-/WPH mixtures was measured using the Nano-ZS (Malvern, Germany) using Smouluchwski mode. The software could determine the suitable type of measurements after obtaining the sample conductivity using the voltage of about 150 V. Samples were measured in triplicate with 10 runs for each measurement.

### 2.3.4 Microstructure Analysis

A small aliquot of binary complex solutions was transferred to a glass microscope slip and covered with a glass coverslip. The optical microscopy images were observed by the light microscope (Leica DM IL LED, Buffalo Grove, IL, USA) equipped with a camera (Vision Research, AMIETEK, Miro Lab 3a10) under a 20× magnification lens (Leica HI Plan, Buffalo Grove, IL, USA). The images were analyzed by the Image J software (version 1.52a, NIH, USA).

The scan electron microscopy of complex solutions was measured using the Field-emission scanning electron microscope (SEM) (Zeiss Gemini 500, Jena, Germany). An aliquot (~10 μL) of the solution was dropped on the pin stub with carbon tape and then vacuum dried overnight in the desiccator. Samples were coated with Au/Pd in a sputter coater (Denton Desk V, NJ, USA) before being scanned and photographed by a high efficiency secondary electron detector with a 20.0 μm aperture. The accelerating voltage was 1 kV.

### 2.3.5 Quantification of LF by HPLC

The HPLC method was developed to quantify the LF in binary complex solutions or after thermal treatment. A reversed-phase HPLC was performed on Agilent 1100/1200 series HPLC systems (Agilent Technologies, CA, USA), equipped with a diode array detector and ChemStation data acquisition program. Detection was carried out at 214 nm. LF separation was performed using the BioZen Intact XB-C8 column (150×4.6 mm, 3.6 μm; Phenomenex, Torrance CA, USA) at 40 °C. A gradient elution was performed using 0.1% trifluoroacetic acid (TFA) in water (mobile phase A) and 0.1% trifluoroacetic acid in Acetonitrile (mobile phase B) at a mobile phase flow rate of 1.0 mL/min using the following gradient elution: 0-5 min, 5% B; 5-20 min, 5-20% B; 20-25 min, 50-5% B. The injection volume was 10 μL. The concentration of remaining native LF in sample solutions can be measured and quantified according to a standard curve of native LF with a concentration of 0-0.2% (w/v) (R²>99%).

### 2.3.6 Complexation Efficiency, LF Loading Ratio, and LF Retention Measurement

The supernatant obtained after centrifugation of the mixtures was appropriately diluted and used to quantify the free LF concentration by HPLC analysis. The complexation efficiency was calculated according to the following equation (Eq.2):

$$Complexation efficiency ( CE ) \% = \frac{Total LF - Free LF}{Total LF} \qquad ( Eq . 2 )$$

Total LF was the theoretical concentration (w/v) of LF employed in the mixture; free LF was the measured concentration (w/v) of LF in the supernatant.

The loading ratio (i.e., mass ratio) of LF in the freeze-dried complex samples was quantified in the redispersed complex samples in 10 mM phosphate buffer pH 7 at a concentration of 0.2% (w/v) using HPLC analysis. The loading ratio of LF in the freeze-dried complex was calculated according to the following equation (Eq.3):

$$Loading ratio of LF = \frac{Concentration of LF in complex solution ( \% , w / v )}{Complex concentration ( 0.2 \% , w / v )} \qquad ( Eq . 3 )$$

The LF retention ratio, which indicates how much LF remains native after thermal treatment, was calculated using the following equation (Eq.4):

$$LF retention ( \% ) = \frac{Concentration of LF after heating}{Concentraiton of LF before heating} . \qquad ( Eq . 4 )$$

### 2.3.7 Electrophoresis Analysis

Unheated and heated pure LF or LF/WPH complex solutions were analyzed using sodium dodecyl sulfate (SDS)-PAGE in a vertical mini gel electrophoresis system (Mini-PRO-TEAN Tetra cell, Bio-Rad, USA). The premixed TGA fast Cast Acrylamide starter kit was used for the preparation of PAGE gels. Twenty microliters of diluted samples (2 mg/mL of protein) were mixed with 2× Laemmli buffer at the ratio of 1:1 and then incubated at room temperature for 3 to 4 hours. Then 20 μL of mixtures were loaded on the gels for electrophoresis (200 V) for about 30 to 45 minutes. The gel was stained in 0.15% (w/v) Coomassie Brilliant R-250 solution which consisted of 50% (v/v) methanol and 10% (v/v) acetic acid for 30 minutes. Then the gel was de-stained in de-staining solutions (20% (v/v) methanol and 10% (v/v) acetic acid) for 24 hours.

### 2.3.8 Thermal Stability Test of LF in Complex

The thermal stability of LF was tested and compared in pure LF and re-dispersed LF/WPH complex solutions in 10 mM phosphate buffer at pH 7 (0.2% w/v). The solution samples (2 mL) were loaded into glass tubes and placed into a water bath at different temperatures of 75 °C and 90 °C for 2 minutes and then immersed in an ice-water bath for several minutes until the samples were cooled to ambient temperature (T=25 °C) before further analysis. The optical images, turbidity, and particle size of complex solutions before and after thermal treatment were measured. HPLC analyses were used to quantify the loading ratio of LF and LF retention rate after thermal treatment. SDS-PAGE images of LF and LF binary complex samples before and after heating were compared.

### 2.3.9 Antimicrobial Activity Analysis

A strain of Staphylococcus aureus and E. coli was used in this study as a target for gram-positive and gram-negative bacteria, respectively. Both LB broth and agar medium were prepared and autoclaved at 121 °C for 15 minutes. The Staphy. aureus or E. coli were activated by being cultured on LB agar medium for 24 hours. A loop of a pure colony was transferred and incubated into a 10 mL fresh LB medium for 24 hours at 37 °C. Generally, the antibacterial activity of LF and LF binary complex against bacteria was performed on a 96-well microtiter plate using the UV absorbance method. First, the bacteria were diluted in LB broth 1000 times to make sure the absorbance of 100 μL bacteria broth was less than 0.04 at 625 nm. The increase in absorbance at 625 nm (OD625 nm) was used to indicate the growth of bacteria.

For the MIC (minimal inhibitory concentration) study, LF at different diluted concentrations (0.1-1%, w/v) was used. A volume of 100 μL diluted S. aureus broth and 100 μL LF solution was added to each well. Then, 100 μL bacteria broth with 100 μL PBS buffer was applied as the control. The minimal concentration of LF to inhibit 50% of bacterial growth (i.e., 50% of OD value in control) was referred to as MIC of LF on inhibiting bacteria and was used as the LF concentration for the further antibacterial study of the LF-WPH complex study. Similarly, 100 μL of diluted bacteria broth and 100 μL of unheated and heated LF-WPH complex solution at the selected concentration (0.2%, w/v) were added to each well. The microtiter plate was incubated at 37 °C and the OD625 nm was measured to monitor the growth of bacteria at 0, 24, and 48 hours of incubation, with shaking 10 seconds before reading.

### 2.4 Data Analysis

The obtained data were presented as means and standard deviations of duplicates or triplicates and analyzed using Analysis of Variance (ANOVA). The difference between mean values was evaluated using the Tukey HSD comparison test (P<0.05). All the statistical analyses were performed using JMP Pro15 (SAS Institute, USA) and plotted by GraphPad Prism9 (GraphPad Software Inc., USA).

### 3. Results and Discussion

### 3.1 Effect of pH and Ratio on the Formation of LF/WPH Complexes

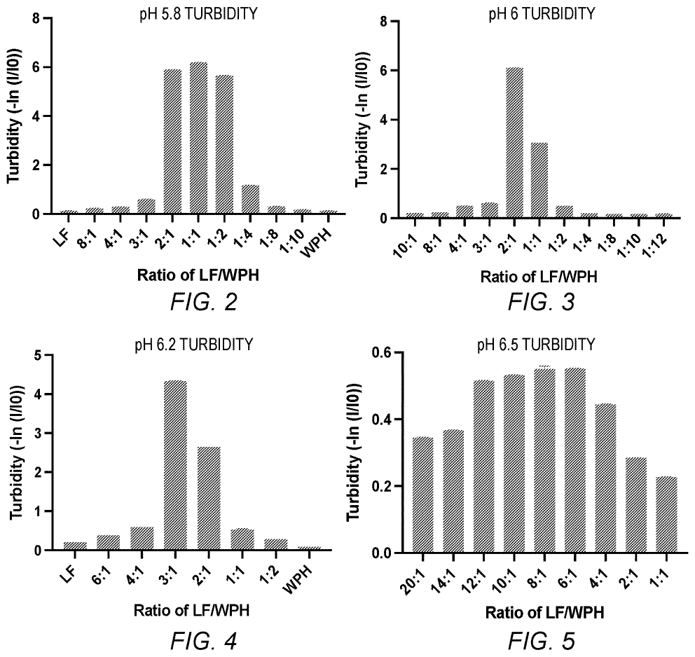

Turbidity is an important indicator to determine whether the complex is formed or not. FIGS. 2-5 show the effect of different LF and WPH mixing ratios on the turbidity of LF/WPH mixtures at different pH conditions. Solutions with a higher turbidity are generally indicative of higher levels of complexation formation. As the pH increases, the turbidity of the LF/WPH mixture decreases and a higher ratio of LF is required to form a higher turbidity solution. As shown in FIG. 2, at pH 5.8, the LF/WPH mixture with a ratio of 1:1 has the highest turbidity; as shown in FIG. 3, at pH 6.0, the LF/WPH mixture with a ratio of 2:1 has the highest turbidity; as shown in FIG. 4, at pH 6.2, the LF/WPH mixture with a ratio of 3:1 has the highest turbidity; and as shown in FIG. 5, at pH 6.5, the LF/WPH mixture with a ratio of 6:1 has the highest turbidity. In addition, FIGS. 2-5 show that the LF/WPH complexes can be formed most remarkably at pH 5.8 and pH 6.0 at their optimal ratios of 1:1 and 2:1, respectively.

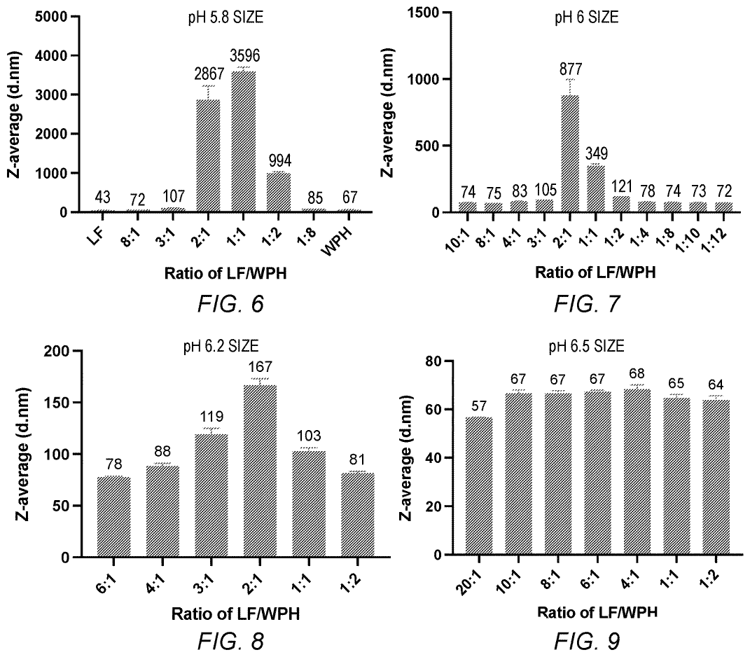

FIGS. 6-9 show the effects of different pH conditions and different LF and WPH mixing ratios on the mean particle size distribution of LF/WPH mixture solutions, the results of which are consistent with the turbidity study of FIGS. 2-5. A larger size of LF/WPH complex was reached with a decrease in pH. As shown in FIG. 6, the mean size of LF is around 10 nm and the mean size of WPH is around 40 nm. The change in pH does not affect the mean size of LF and WPH. As a result, the abrupt increase in the particle size (e.g., diameter) of LF/WPH mixtures under optimal conditions (i.e., pH 5.8 at 1:1 and 2:1 ratio of LF/WPH [FIG. 6] and pH 6 at 1:1 and 2:1 ratio of LF/WPH [FIG. 7]) can be explained by the formation of complexes.

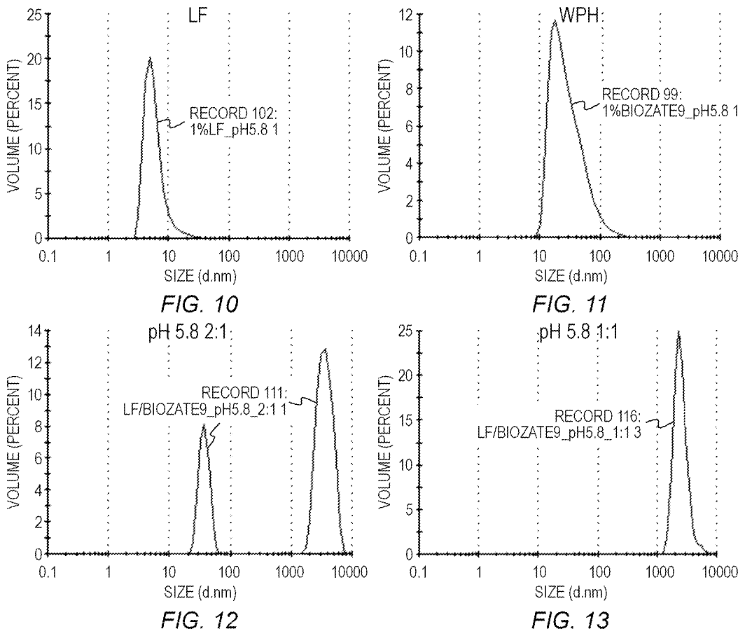

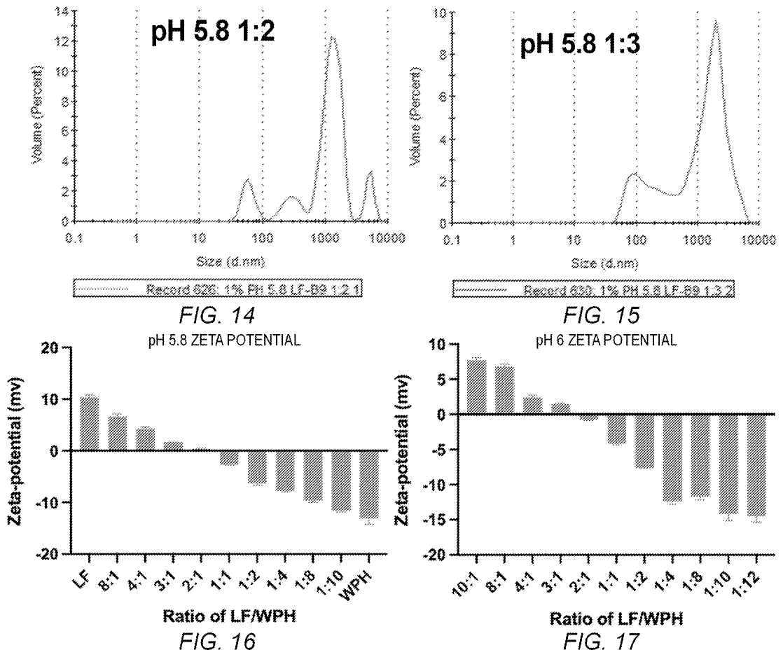

The particle size distribution of LF, WPH, and LF/WPH mixture solutions at pH 5.8 is further illustrated in FIGS. 10-15. LF and WPH showed a mono peak at a size range of 1-100 nm (FIG. 10) and 10-500 nm (FIG. 11), respectively. As shown in FIG. 12, LF/WPH mixtures formed at pH 5.8 and a ratio of 2:1 (LF:WPH) showed two peaks corresponding to free LF biopolymers (5-100 nm) and LF/WPH complexes (1000-10000 nm). In contrast, the LF/WPH mixtures formed at pH 5.8 and a ratio of 1:1 (LF:WPH) showed almost exclusively complex with a mono peak at 1000-10000 nm (FIG. 13). The LF/WPH mixtures formed at pH 5.8 and ratios of 1:2 (FIG. 14) and 1:3 (FIG. 15) showed multiple peaks due to excessive free biopolymers in the systems.

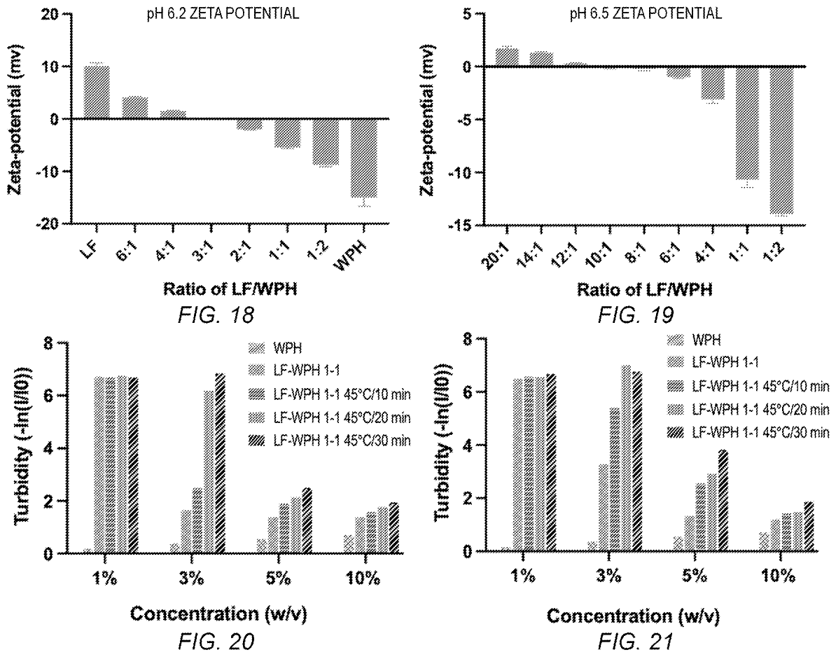

FIGS. 16-19 show the zeta potential of LF/WPH mixtures at different pH conditions and different LF and WPH mixing ratios. The pI of LF is about 8.2, so it will carry a positive charge in an acidic environment, while the pI of WPH is about 4.5, so it will carry a negative charge when pH is higher than 4.5. When two polymers carry opposite charges, the polymers are able to form complexes due to electrostatic interaction. For LF and WPH, the optimal pH range for the formation of LF/WPH complexes is between pH 5.8 and pH 6.5. When the zeta-potential of LF/WPH mixing solution is close to 0, it leads to a large amount of LF/WPH complex formation with highest turbidity at the ratio of 1:1 and 2:1. This confirmed the hypothesis that the major driving forces for the complexation between LF and WPH are electrostatic interactions.

The effect of total concentration (1%-10%), mixing temperature, and time on complex formation at selected mixing ratios of 1:1 and 1:2 (LF:WPH) were further investigated, as shown in FIGS. 20-25. Upon mixing at room temperature for 30 minutes, regardless of the LF:WPH ratio, 1% mixtures presented a large complexation as indicated by high turbidity (<6) and large mean particle size (>6,000 nm). However, as shown in FIGS. 20-23, when the concentration increased to 3%, 5%, and 10%, the turbidity of the mixtures decreased (between 0-4) and the mixtures exhibited a smaller mean particle size (<200 nm). Without wishing to be bound to any particular theory, the increase in concentration may cause the distance between same molecules (LF and LF or WPH and WPH) to increase and the distance between different molecules (LF and PH) to decrease, thus decreasing intermolecular interactions between LF and WPH. Such a decrease in interactions and complexation in higher concentrations could further be explained by zeta potential charges. As shown in FIGS. 24-25, the absolute charges of LF and WPH both decreased with the increase in concentration. This indicates that the surface charges of the biopolymers decreased, which may lead to a decrease in electrostatic interactions.

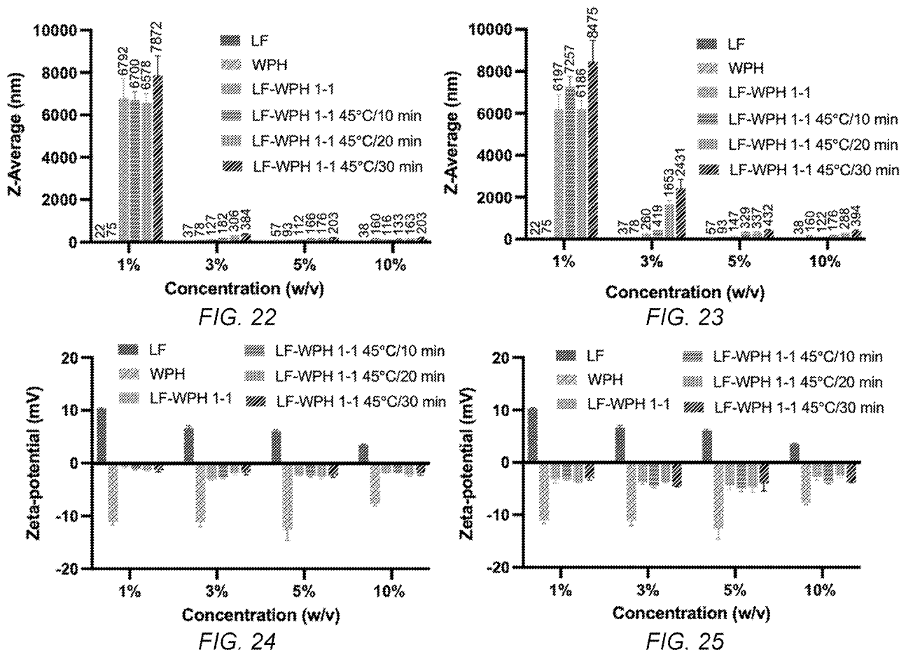

To further investigate whether a higher mixing temperature could promote complexation, mixture solutions were incubated in a water bath at 45 °C for 10, 20, and 30 minutes. The impact of incubation on 1% samples was minimal, as these samples already formed large complexes without incubation. For higher concentrations, incubation at 45 °C did promote more complexation, as mixtures showed an increased turbidity and particle size. Furthermore, the longer the incubation time, the more complexation was induced. Particularly, after being incubated for 30 minutes, 1:1 and 1:2 (LF:WPH) mixtures at 3% concentration reached a turbidity of 6, similar to the turbidity of 1% mixtures (FIGS. 20 and 21). The mean size of 1:1 and 1:2 (LF:WPH) mixtures at 3% concentration after 30 minutes of incubation was shown to be 384 nm and 2,431 nm, respectively (FIGS. 22 and 23). Although the mixtures at concentrations of 5% and 10% also demonstrated an increased turbidity and particle size after mixing for 30 minutes, such effect was less significant as compared to the samples at a 3% concentration. Therefore, the chosen complex formation conditions are a concentration of 1% mixing at room temperature for 30 minutes and a concentration of 3% mixing at 45 °C for 30 minutes. Considering the high complexation and turbidity formed at a concentration of 1%, those samples were further studied regarding yield, complexation efficiency, structures, and thermal stability.

### 3.2 Yield and Complexation Efficiency of LF/WPH Complex

The yield, complexation efficiency, and loading ratio of LF/WPH complex formed at pH 5.8 was calculated (Table 1). Based on the results of turbidity (FIGS. 2-5) and particle size (FIGS. 6-9) measurements, freeze-dried LF/WPH complexes were prepared under the ratio of forming the LF/WPH complex at certain pH conditions after the centrifuge process. Table 1 shows that the mixture of LF and WPH at a 1:1 ratio at pH 5.8 could reach a high yield of about 40% among all studied conditions. A higher complexation efficiency was observed at pH 5.8 at the LF/WPH ratio of 1:1 and 1:2. This result is consistent with the turbidity and particle size measurements. Generally, the loading ratio of LF in the final freeze-dried samples is consistent with the mass ratio of LF when preparing the mixture solutions. For example, as shown in Table 1 at a 1:1 ratio, LF has a final loading ratio of 48%, which is close to the mass ratio of LF (50%) in the initial mixture samples.

TABLE 1
The yield, complexation efficiency (CE), loading ratio (LR) of
formed LF-WPH complex at pH 5.8 with different LF/WPH ratios.

| Ratio of | | | |
|---|---|---|---|
| LF/WPH | | | |
| (pH 5.8) | Yield (%) | CE(%) | LR(%) |
| 2:1 | 25.33 ± 0.05 | 19.94 ± 0.30 | 60.27 ± 0.03 |
| 1:1 | 40.14 ± 2.24 | 46.81 ± 1.55 | 48.26 ± 0.20 |
| 1:2 | 36.33 ± 3.35 | 46.01 ± 3.57 | 43.25 ± 0.12 |
| 1:3 | 16.89 ± 1.26 | 10.02 ± 3.72 | 31.06 ± 0.33 |

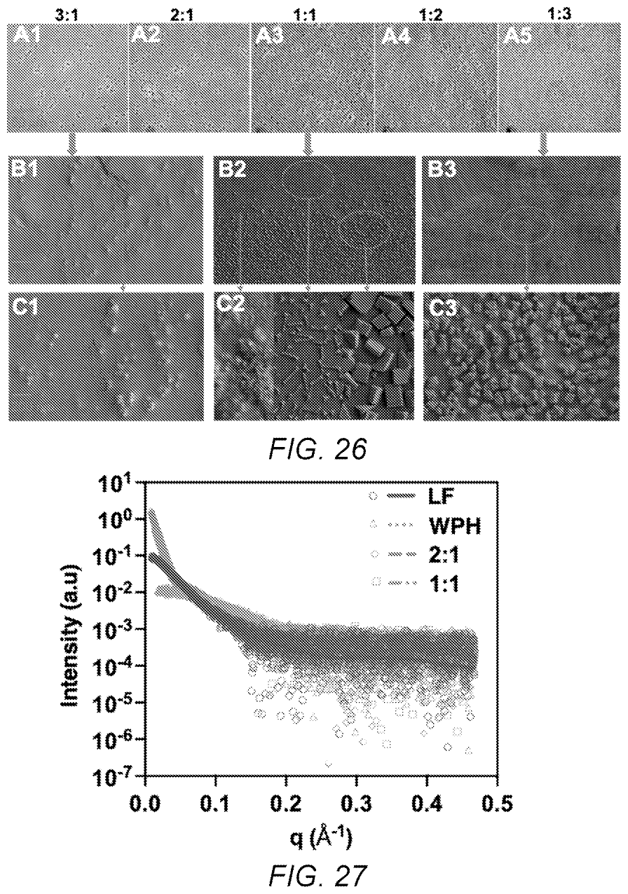

As shown in FIGS. 33-34, the optical microscopy of LF/WPH mixtures formed at a ratio of 2:1 (LF:WPH) at pH 5.8 (FIG. 33) and at a ratio of 1:1 (LF:WPH) at pH 5.8 (FIG. 34) showed that a large amount of protein-peptide complex was formed and then sedimented on the bottom of the mixture following overnight setting at 4 °C, showing amorphous interpolymeric complex structures. The optical microscopy and scan electron microscopy of LF-WPH mixtures at pH 5.8 and different LF:WPH ratios are shown in FIG. 26. At a ratio of 1:1 (LF:WPH; FIG. 26, Panels A3 and B3) and 1:2 (LF:WPH; FIG. 26, Panel A4), more and larger complex particles were shown compared to other ratio conditions. In general, the protein-peptide complex showed as amorphous interpolymeric complex structures. After air-drying, complex particles demonstrated different morphology and sizes in different mixing ratios. Due to limited complexation at a 3:1 ratio, only small complexes were formed with a size of about 100-200 nm (FIG. 26, Panels B1 and C1). Interestingly, in the 1:1 ratio samples, the complexes formed into three categories of shape: loose matrix, rod-like, and cube-like (FIG. 26, Panels B2 and C2). The loose matrix particles were more likely individual free biopolymers without complexation, while the rod-like and cube-like particles were complexes having different densities. However, when the mixing ratio shifted to 1:3, only small cube-like particles presented with a size of 100-200 nm, showing as exclusively nanocomplexes (FIG. 26, Panels A5, B3, and C3). The negative charges in the 1:3 ratio conditions may prevent the further aggregation of small complexes into large complexes, as happened in the 1:1 ratio conditions.

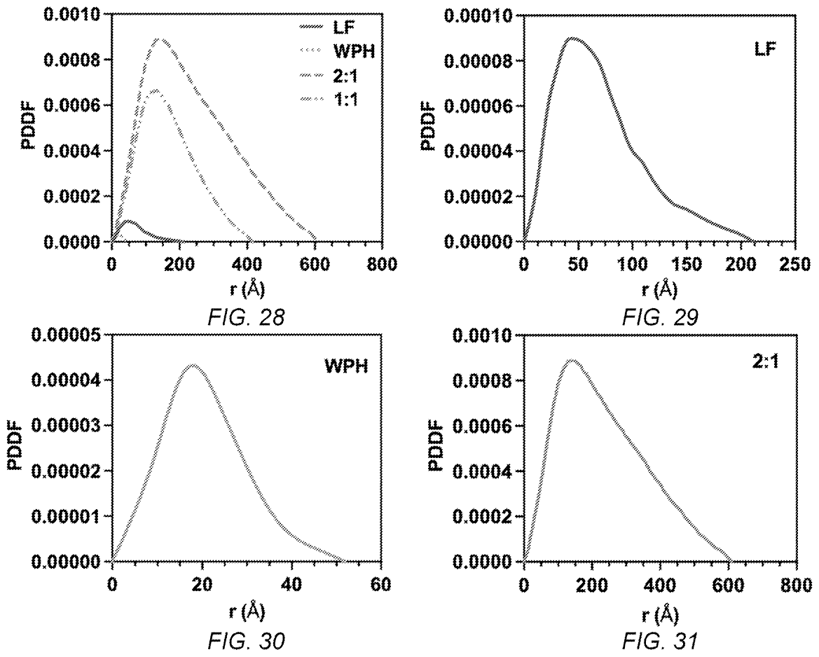

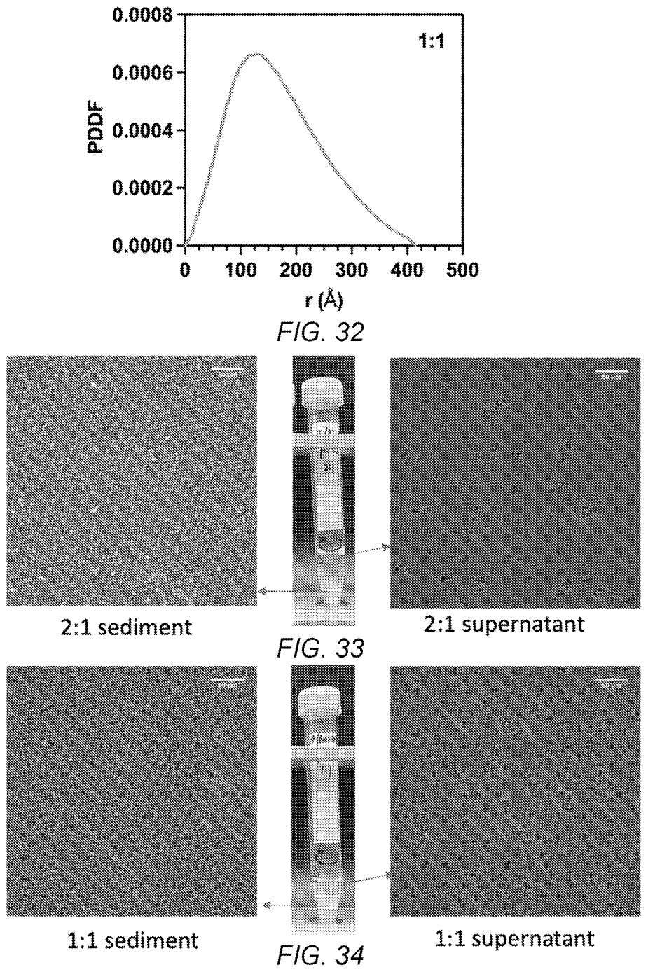

Small angle-X-ray Scattering (SAXS) was further applied to provide molecular structural and conformational information of individual biopolymers and complexes (FIGS. 27-32). The obtained SAXS data of LF are comparable with previous studies (FIG. 27). The pair-distance distribution functions (PDDF) of all samples are shown in FIGS. 28 and 29. Individual LF showed a radius of gyration of about 5.7 nm, which is slightly larger (R_{g}=4.2 nm) than that reported in other studies. This difference may be related to the measurement pH conditions. In the current study, LF was measured at pH 5.8, resulting in more open structures compared to that measured at a pH of 7 reported in other studies. The largest intermolecular distance (Dmax) was 21.2 nm (FIG. 29). The shape of the PDDF graph indicated that LF was not shown as a globular structure, but rather an elongated structure made up of two globular lobes. WPH as hydrolyzed peptides had a smaller radius of gyration and Dmax than that of LF (FIG. 30). Although composed of a mixture of different peptide chains, the WPH seemed to mainly present as a globular structure. For the LF-WPH complex at pH 5.8, those formed at a 2:1 ratio showed a slightly larger Rg and Dmax which indicated larger complex dimensions than those formed at a 1:1 ratio (FIGS. 31 and 32).

### 3.3 Effect of LF/WPH Complex Formation on the Thermal Stability of LF

LF is prone to denaturation during heat treatment, especially under a neutral pH environment when the pH is close to the pI of LF. To test whether the formation of the LF/WPH complex could improve the thermal stability of LF, both the centrifuged, freeze-dried LF/WPH complex and uncentrifuged, direct freeze-dried (DF) samples formed at their optimal pH (5.8) from 2:1 to 1:3 ratios were dissolved in 10 mM phosphate buffer at pH 7 at 0.2% (w/v) and then heated at water bath at 75 °C/2 min and 90 °C/2 min. The stability could be qualitatively indicated by an increase in both turbidity and particle size, as denatured protein will aggregate and form large particles. It can also be quantitively measured by using HPLC analysis to calculate the concentration of native LF retained in the thermally treated samples.

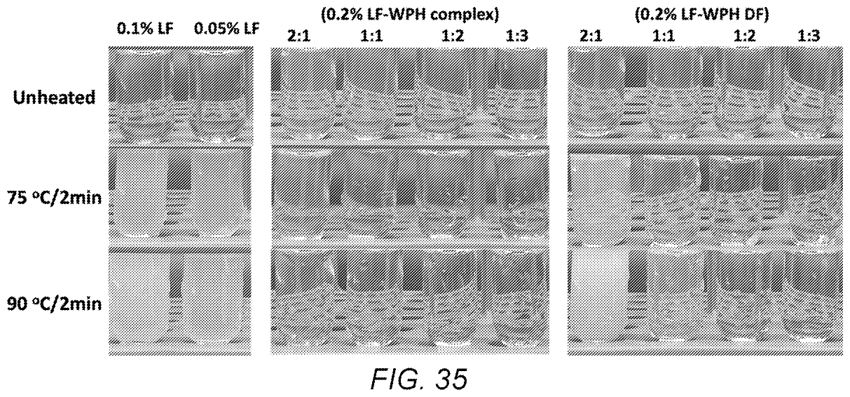

FIG. 35 shows optical images of pure LF, centrifuged, freeze-dried LF/WPH complex, and uncentrifuged, direct freeze-dried LF/WPH complex samples in phosphate buffer (10 mM, pH 7) before and after thermal treatment. FIG. 35 shows that pure LF solutions tended to aggregate and exhibit increased turbidity after being heated. However, all centrifuged, freeze-dried LF/WPH complexes remained as clear solutions with low turbidity, even after being heated up to 90 °C for 2 minutes. For the uncentrifuged, direct freeze-dried samples, at increased ratios of LF, especially at the ratios of 2:1 and 1:1, the solutions became turbid after thermal treatment. This can be expected because the uncentrifuged, direct freeze-dried samples still contained free, un-complexed LF in the samples which would not be protected from thermal aggregation.

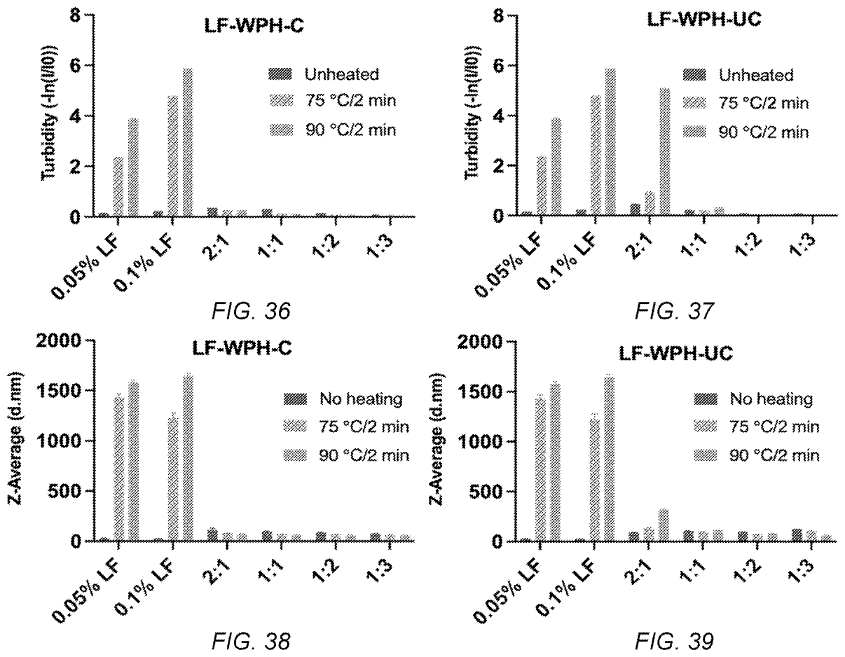

The turbidity and particle size of unheated and heated samples were further quantitatively measured to confirm whether protein aggregation occurred during heating. As shown in FIGS. 36-39, pure LF solutions showed a remarkable increase in turbidity and mean particle size at both concentrations of 0.1% and 0.05%. However, the centrifuged, freeze-dried LF/WPH complex showed a limited change in turbidity and mean particle size after thermal treatment, regardless of LF/WPH ratio, as shown in FIG. 36. This indicates a significant improvement in the thermal stability of LF when present in a centrifuged, freeze-dried LF/WPH complex. As shown in FIG. 38, only the 2:1 ratio of the uncentrifuged, direct freeze-dried LF/WPH sample (LF/WPH DF sample) exhibited a large increase in turbidity and mean particle due to a larger ratio of un-complexed LF present in the samples. However, the other LF/WPH DF sample ratios exhibited an un-detectable change in turbidity and mean particle size, indicating a good protection of LF during thermal treatment. These results are consistent with the visual appearance of the LF/WPH DF samples (see FIG. 35).

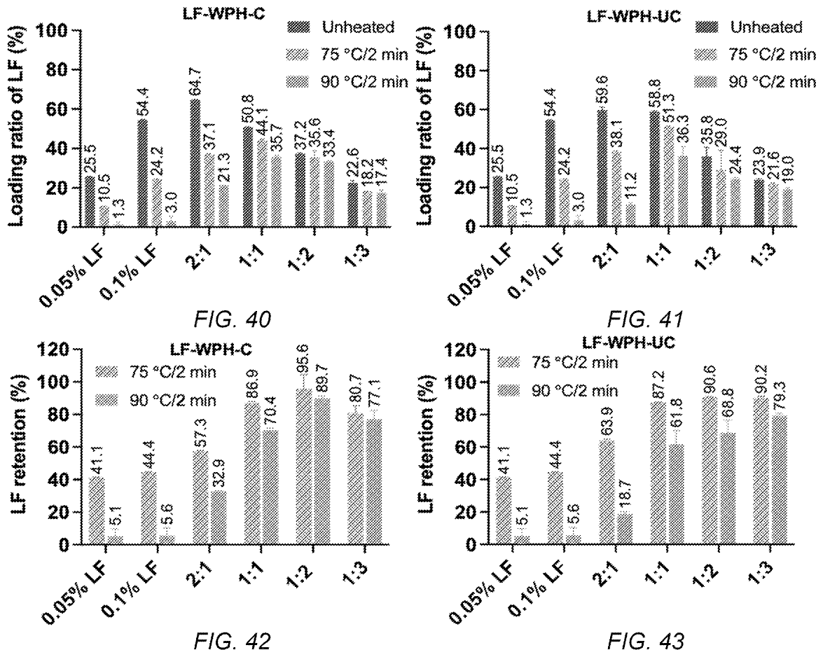

To further confirm and quantify how much LF retained native conformation following thermal treatment, HPLC analysis was used to measure the concentration of LF in the sample solutions before and after heating. FIGS. 40-43 show the LF loading ratio and retention ratio in pure LF, centrifuged, freeze-dried LF/WPH complex, and uncentrifuged, direct freeze-dried LF/WPH (DF) samples in phosphate buffer (10 mM, pH 7) before and after thermal treatment. As expected, with the decreased mass ratio (2:1 to 1:3) of LF, the loading ratio of LF in the complex decreased from about 70% to 20% in both centrifuged, freeze-dried LF/WPH complex and LF/WPH DF samples, as shown in FIGS. 40 and 41. The loading ratio was comparable with the initial mass ratio in the prepared samples. For pure LF solutions, the loading ratio of LF significantly decreased from 25% to 2% and 54% to 5% at the concentration of 0.1% and 0.05%, respectively, after 90 °C/2 min treatment.

As shown in FIGS. 42 and 43, the pure LF retention was around 40% after 75 °C/2 min treatment and only 8% after 90 °C/2 min treatment. After forming a complex with WPH, the LF retention was 85-90% after 75 °C/2 min treatment and 50-80% after 90 °C/2 min treatment in different LF/WPH ratios, which was significantly higher than the LF retention in pure LF solutions. Overall, the centrifuged, freeze-dried LF/WPH complex showed a slightly higher LF retention than uncentrifuged, direct freeze-dried LF/WPH (DF) samples. Although samples at the ratio of 1:3 showed the highest LF retention overall, the initial mass ratio of LF in the prepared samples was the lowest (20-24%). Considering both the loading ratio and the retention of LF, the ratio of 1:1 was the optimal condition with a relatively high LF mass ratio (35-50%) and high LF retention (60-87%) in unheated and heated samples.

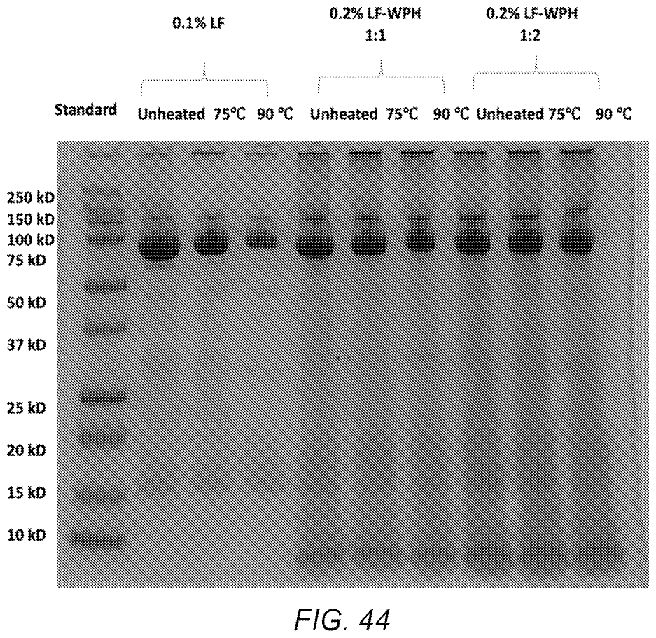

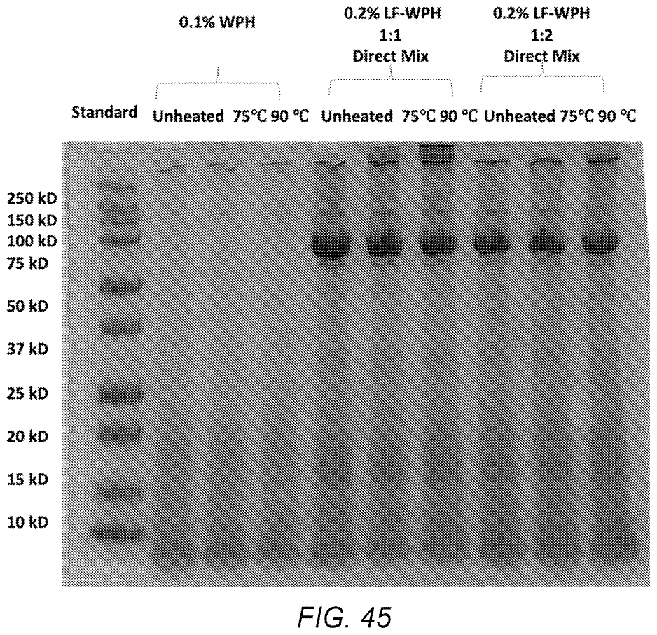

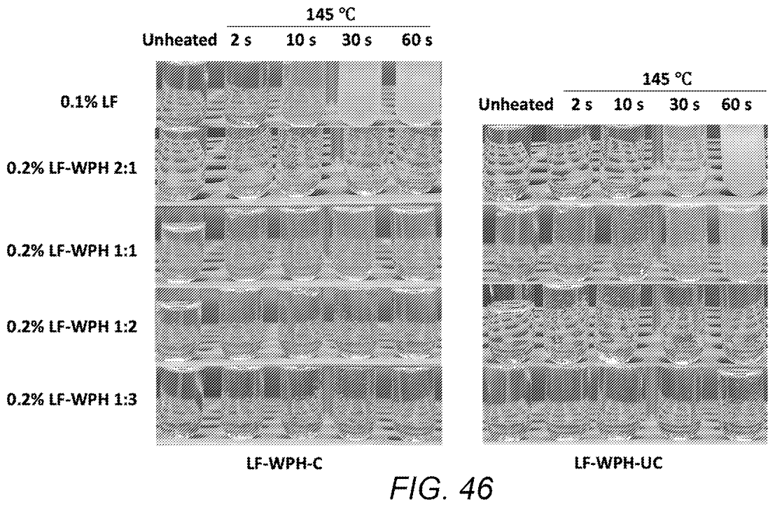

The high LF retention after heating treatment was also confirmed by the SDS-PAGE analysis. As shown in FIGS. 44-45, SDS-PAGE analysis was conducted on unheated and heated (75 °C/2 min or 90 °C/2 min) samples of pure LF, re-dispersed LF/WPH complex (FIG. 44), and LF/WPH direct mixture (FIG. 45) in phosphate buffer (10 mM, pH 7). As shown in FIG. 44, LF in the formed LF/WPH complex showed neglectable changes in the band (around 75 kDa) density during thermal treatment, which indicates a good LF retention and thermal stability in LF/WPH complex samples. However, as shown in FIG. 45, the LF in direct mixtures of LF and WPH showed a lighter band, indicating a lower LF retention and thermal stability.

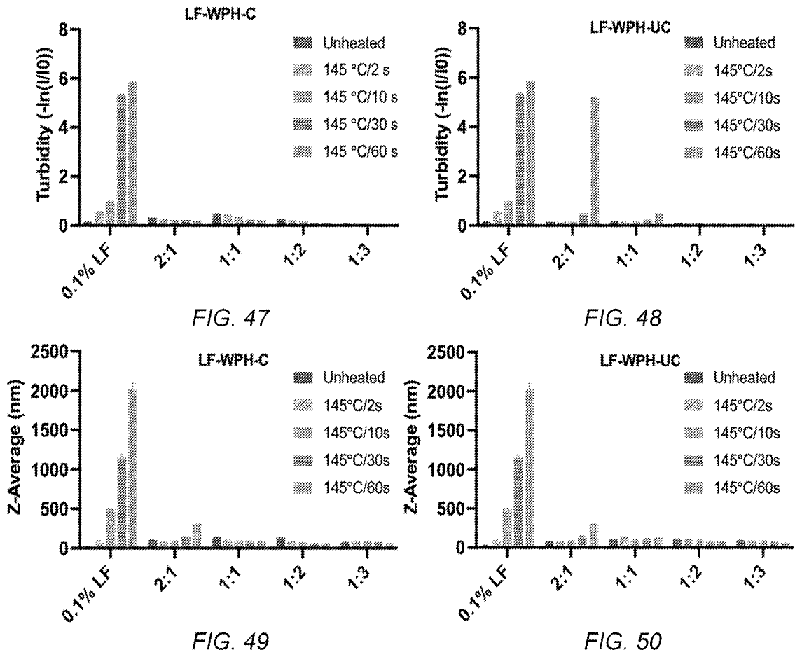

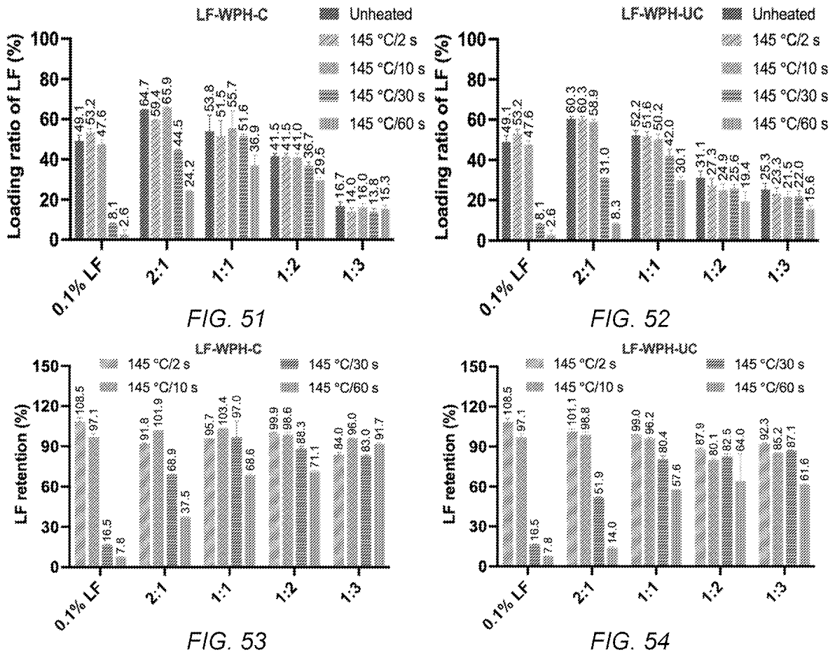

The thermal stability of LF, LF/WPH complex samples, and uncentrifuged LF/WPH complex samples were further tested in oil bath conditions at 145 °C for 0-60 seconds to mimic high-temperature short-time (HTST) processing treatments. The optical images, turbidity, and particle size changes of all samples before and after oil bath heating are shown in FIGS. 46-50. Pure LF samples started to show protein aggregation and a cloudy appearance after 10 seconds of oil bath heating, and turbidity and particle size significantly increased after 30 seconds of oil bath heating. However, all LF/WPH complex samples displayed limited turbidity and particle size changes even after 60 seconds of heat treatment, indicating an enhanced thermal stability (FIGS. 46-50). For uncentrifuged complex samples, those formed at a ratio of 2:1 and 1:1 exhibited an increased turbidity, indicating a lower thermal stability than the 1:2 and 1:3 ratio samples (FIGS. 48 and 50). This is due to the larger amount of un-complexed LF in the 2:1 and 1:1 ratio samples. HPLC analysis on the LF retention further confirmed that, under oil bath heating conditions for up to 30 seconds of treatment, all complex samples, whether centrifuged or uncentrifuged, exhibited a high LF retention (>80%), whereas pure LF only retained 16.5% native protein (FIGS. 51-54).

### 3.3 Antibacterial Activity of LF/WPH Complex

The antimicrobial properties of LF were evaluated in pure and complex LF samples before and after thermal treatment. This part of the study aims to examine whether complexation will influence the functionality of LF and preserve this capacity after heating treatment. Antimicrobial capacity was chosen as a representative biological functionality of LF, as it can be easily performed and is relatively safe in most biological labs. A strain of Staphylococcus aureus and Escherichia coli were tested as the representatives of gram-positive and gram-negative bacteria, respectively.

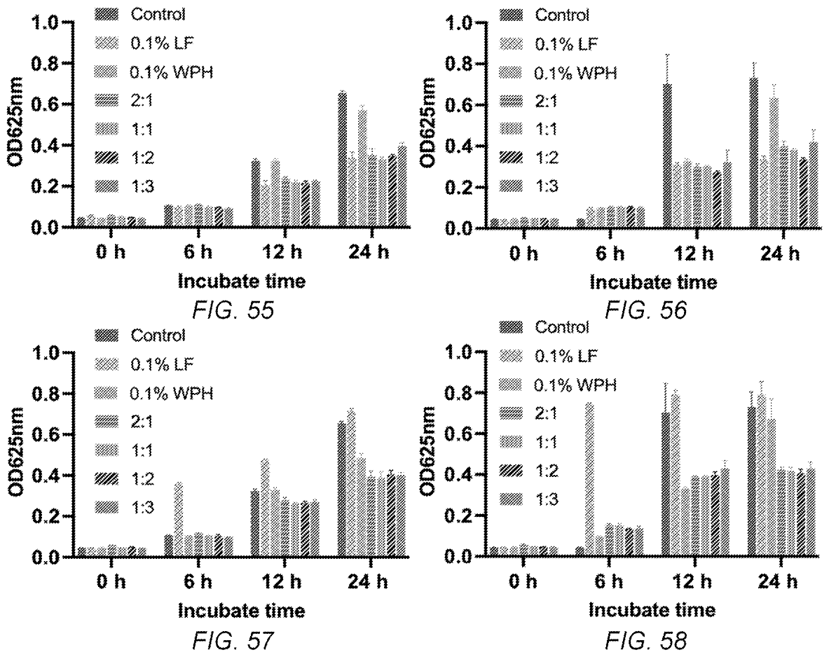

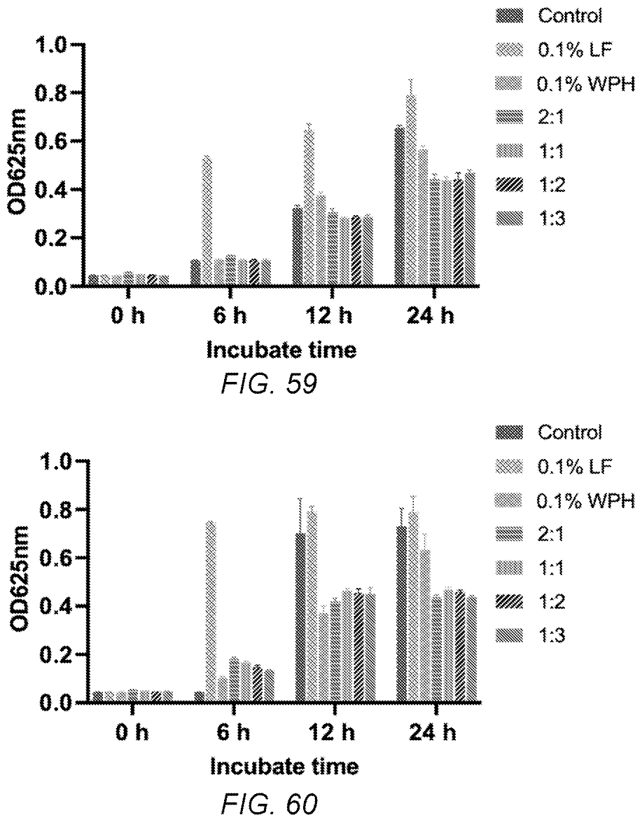

As shown in FIGS. 55 and 56, after 24 hours of incubation, both pure LF and LF/WPH complex inhibited the bacteria growth by half while WPH did not significantly inhibit the bacterial growth, which means that the antibacterial effect of the LF/WPH complex was mainly from LF rather than from WPH. FIGS. 57-60 show that, after being heated at 75 °C/2 min and 90 °C/2 min, pure LF lost its antibacterial properties on both S. aureus and E. coli as it showed similar OD625 nm values as the control. For the LF/WPH complex, the OD625 nm value was slightly increased after 75 °C/2 min (FIGS. 57 and 58) and 90 °C/2 min (FIGS. 59 and 60), compared to the unheated LF/WPH complex. However, all of the LF/WPH complex samples still demonstrated a significant antibacterial effect compared to the control, even after being heated up to 90 °C/2 min. This result indicates that the antibacterial activity of LF was well retained in the LF/WPH complex even after thermal treatment.

### Example 2

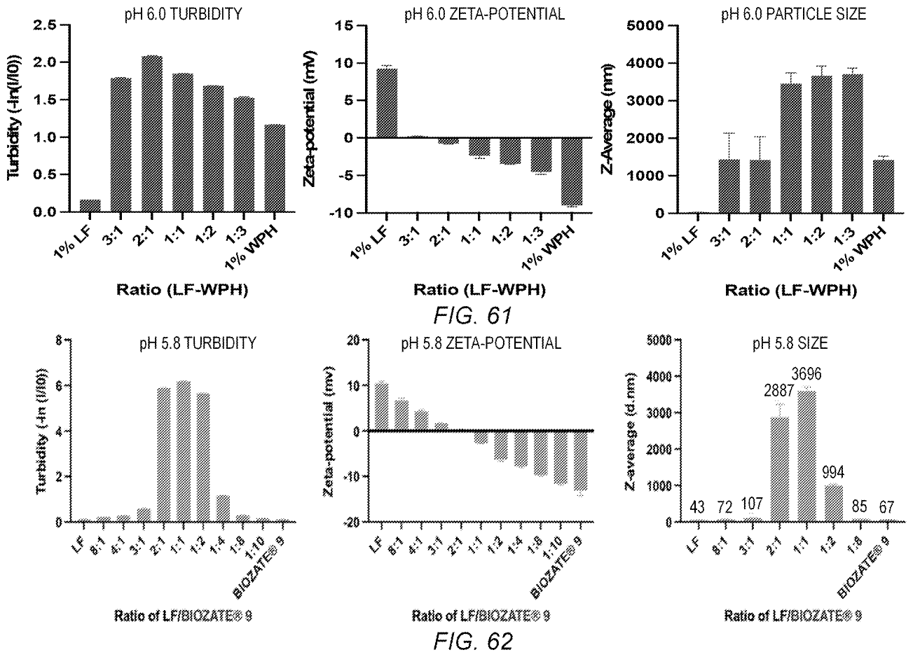

Complex formation between LF and a WPH that was hydrolyzed with chymotrypsin was tested. Specifically, whey protein isolate (WPI) was enzymatically hydrolyzed with chymotrypsin at a concentration of 5% by weight of the WPI for 24 hours at 50 °C to provide a degree of hydrolysis of 15%-20%. Complex formation between LF and a commercially available whey protein hydrolysate BIOZATE® 9 (available from Agropur) was also tested. The complexes were formed as described in Example 1. The results for turbidity, zeta-potential, and average particle size for the resulting complexes at various ratios of LF:WPH are provided in FIG. 61 (LF-WPH hydrolyzed with chymotrypsin) and FIG. 62 (LF-BIOZATE® 9). Without wishing to be bound to any particular theory, the different complexation results between these two kinds of WPH may be related to the differences in zeta-potential (WPH hydrolyzed with chymotrypsin has a lower zeta-potential (e.g., greater than −10 mV) than BIOZATE® 9 (e.g., less than −10 mV)), differences in particle size (WPH hydrolyzed with chymotrypsin has a mean particle size of greater than 1,000 nm, whereas BIOZATE® 9 has a mean particle size of less than 100 nm), and differences in molecular weight (the molecular weight of BIOZATE® 9 is, in general, lower than that of WPH hydrolyzed with chymotrypsin (e.g., in a BIOZATE® 9 sample having more than 85% is lower than 10 kDa, more than 70% is lower than 5 kDa, and more than 50% is lower than 2 kDa)). Each of these differences may affect electrostatic interactions with lactoferrin, thus influencing complex formation.

The foregoing is illustrative of the present invention, and is not to be construed as limiting thereof. The invention is defined by the following claims, with equivalents of the claims to be included therein. All publications, patent applications, patents, patent publications, and other references cited herein are incorporated by reference in their entireties for the teachings relevant to the sentence and/or paragraph in which the reference is presented.

That which is claimed is:

1. A complex comprising:
a lactoferrin; and
a whey protein hydrolysate,
wherein the lactoferrin and the whey protein hydrolysate are associated via electrostatic interactions.

2. The complex of claim 1, wherein the complex has a zeta potential of about −10, −5, −2, or −1 mV to about +1, +2, +5, +10, or +15 mV, optionally wherein the complex has a zeta potential of about 0 at a pH of about 5.8 to about 6.5.

3. The complex of claim 1 or 2, wherein the complex comprises the lactoferrin in an amount of about 25% to about 75% or 80% w/w and the whey protein hydrolysate in an amount of about 25% to about 75% w/w, optionally wherein the complex comprises the lactoferrin in an amount of about 50% or 65% to about 75% w/w and the whey protein hydrolysate in an amount of about 25% to about 50% w/w or wherein the complex comprises the lactoferrin in an amount of about 50% and the whey protein hydrolysate in an amount of about 50% w/w.

4. The complex of any preceding claim, wherein the lactoferrin has a net positive charge at a pH of less than 4, 5, 6, or 7, optionally wherein the lactoferrin has a zeta potential of greater than about +1 mV to about +20 mV at a pH of about 4, 4.5, or 5 to about 6, 6.5, or 7.

5. The complex of any preceding claim, wherein the lactoferrin is bovine lactoferrin or human lactoferrin and/or wherein the lactoferrin has an amino acid sequence having at least 70%, 75%, 80%, 85%, 90%, 95%, 96%, 97%, 98%, 99%, or more sequence identity to SEQ ID NO:1 and/or SEQ ID NO:2.

6. The complex of any preceding claim, wherein the lactoferrin is soluble in water at a pH of less than 8 and/or at a pH of greater than 9.

7. The complex of any preceding claim, wherein the lactoferrin has a pI of about pH 8.

8. The complex of any preceding claim, wherein the whey protein hydrolysate has a net negative charge at a pH of less than 5, 6, 7, or 8, optionally wherein the whey protein hydrolysate has a zeta potential of about −20 mV to about −1 mV at a pH of about 5, 5.5, or 6 to about 6.5, 7, 7.5, or 8.

9. The complex of any preceding claim, wherein the whey protein hydrolysate is soluble in water at a pH of greater than 5 and less than 8.

10. The complex of any preceding claim, wherein the whey protein hydrolysate has a hydrolysis rate of about 8%, 9%, or 10% to about 11%, 12%, or 13%.

11. The complex of any preceding claim, wherein the whey protein hydrolysate comprises a mixture of peptides and/or amino acids and at least about 75%, 80%, or 85% of the mixture by weight has a molecular weight of less than 10 or 20 kilodaltons (kDa), optionally wherein at least about 60%, 65%, or 70% of the mixture by weight has a molecular weight of less than 5 kDa and/or at least about 40%, 45%, or 50% of the mixture by weight has a molecular weight of less than 2 kDA.

12. The complex of any preceding claim, wherein the whey protein hydrolysate has a particle size (e.g., diameter) in a range of about 1, 50, or 100 nm to about 1 or 2 microns and/or wherein the whey protein hydrolysate has a particle size (e.g., diameter) of less than 300, 200, or 100 nm.

13. The complex of any preceding claim, wherein the whey protein hydrolysate has a pI of about 3 to about 5, optionally wherein the whey protein hydrolysate has a pI of about pH 4.5.

14. The complex of any preceding claim, wherein the lactoferrin and the whey protein hydrolysate are each a food-grade component, optionally wherein the lactoferrin and/or the whey protein hydrolysate are obtained and/or derived from a natural product (e.g., a food, plant, animal by-product (e.g., milk), etc.).

15. The complex of any preceding claim, wherein the complex is an interpolymeric complex, optionally wherein the complex is an amorphous interpolymeric complex structure.

16. The complex of any preceding claim, wherein the lactoferrin has increased stability (e.g., reduced degradation of the lactoferrin) after exposure to a temperature of about 70 °C to about 80 °C, 90 °C, or 100 °C for about 1 minute to about 60 minutes compared to the stability of the lactoferrin alone (i.e., not present in the complex) after exposure to the same temperature for the same period of time.

17. The complex of any preceding claim, wherein the lactoferrin, after exposure to a temperature of about 70 °C to about 80 °C, 90 °C, or 100 °C for about 1 minute to about 60 minutes, is degraded by less than 50% as measured by high-performance liquid chromatography, optionally wherein the lactoferrin, after exposure to a temperature of about 70 °C to about 80 °C, 90 °C, or 100 °C for about 1 minute to about 60 minutes, is degraded by less than 40% as measured by high-performance liquid chromatography.

18. The complex of any preceding claim, wherein, after exposure of the complex to a temperature of about 70 °C to about 80 °C, 90 °C, or 100 °C for about 1 minute to about 60 minutes, the activity of the lactoferrin is increased compared to the activity of the lactoferrin alone after the same exposure conditions.

19. The complex of any preceding claim, wherein the lactoferrin has increased stability (e.g., reduced degradation of the lactoferrin) after exposure of the complex to a temperature of about 145 °C for about 2 seconds to about 60 seconds (e.g., a high-temperature short time (HTST) treatment under oil bath conditions at about 145 °C for about 2 seconds to about 60 seconds) as compared to the stability of the lactoferrin alone (i.e., not present in the complex) after exposure to the same conditions (e.g., the same temperature for the same period of time).

20. The complex of any preceding claim, wherein, after exposure of the complex to a temperature of about 145 °C for about 2 seconds to about 60 seconds (e.g., a high-temperature short time (HTST) treatment under oil bath conditions at about 145 °C for about 2 seconds to about 60 seconds), the lactoferrin is degraded by less than 30%, optionally by less than 20%, optionally as measured by high-performance liquid chromatography.

21. The complex of any preceding claim, wherein the antimicrobial capacity and/or activity of the lactoferrin (e.g., the antibacterial activity of the lactoferrin on Gram-positive and/or Gram-negative bacteria), after exposure of the complex to a temperature of about 70 °C or 75 °C to about 80 °C, 85 °C, or 90 °C for about 30 seconds to about 2 minutes, is retained and/or improved (e.g., increased) as compared to the antimicrobial capacity and/or activity of the lactoferrin (e.g., the antibacterial activity of the lactoferrin on Gram-positive and/or Gram-negative bacteria) alone, optionally after exposure to the same conditions (e.g., the same temperature and temperature exposure time).

22. The complex of any preceding claim, wherein, after ingestion of the complex by a subject, the bioavailability of the lactoferrin is increased compared to the bioavailability of the lactoferrin after ingestion by a subject.

23. The complex of any preceding claim, wherein the complex, optionally in a liquid composition, has an average size (e.g., diameter) of about 300, 500, 1000, or 1500 nm to about 2000, 2500, 3000, 3500, or 4000 nm.

24. The complex of any preceding claim, wherein the complex has a loose matrix structure, a rod-like structure, and/or a cube-like structure.

25. The complex of any preceding claim, wherein the complex is devoid of a cationic and/or an anionic biopolymer (i.e., the complex is devoid of a biopolymer that is cationic and/or anionic that is not a lactoferrin or a whey protein hydrolysate).

26. A composition comprising the complex of any one of claims 1-25.

27. The composition of claim 26, wherein the complex is in the form of a particle, optionally wherein the particle is a freeze-dried, spray-dried, and/or the like particle.

28. The composition of claim 26 or 27, wherein the composition comprises a plurality of particles, optionally wherein the composition is in the form of a powder (e.g., a dry powder).

29. The composition of any one of claims 26-28, wherein the composition is an aqueous composition and the complex is dissolved and/or suspended in the composition.

30. The composition of any one of claims 26-29, wherein the particle is prepared and/or isolated from a solution comprising less than 6%, 5%, 4%, 3%, 2%, or 1% w/v free lactoferrin (i.e., lactoferrin that is not associated with whey protein hydrolysate via electrostatic interactions).

31. The composition of any one of claims 26-30, wherein the composition and/or plurality of particles comprise less than 5% by weight free lactoferrin, optionally wherein the composition and/or plurality of particles comprise less than 1% by weight free lactoferrin.

32. The composition of any one of claims 26-31, wherein a physiochemical property of the complex (e.g., turbidity and/or particle size), after exposure of the composition to a temperature of about 70 °C or 75 °C to about 80 °C, 85 °C, or 90 °C for about 30 seconds to about 2 minutes, remains within about +30% of the original physiochemical property of the complex prior to the exposure (e.g., the physiochemical property of the complex at initial formation of the complex and/or immediately prior to the exposure).

33. The composition of any one of claims 26-32, wherein a physiochemical property of the complex (e.g., turbidity and/or particle size), after exposure of the composition to a temperature of about 145 °C for about 2 seconds to about 60 seconds (e.g., a high-temperature short time (HTST) treatment under oil bath conditions at about 145 °C for about 2 seconds to about 60 seconds), remains within about +30% of the original physiochemical property of the complex prior to the exposure (e.g., the physiochemical property of the complex at initial formation of the complex and/or immediately prior to the exposure).

34. A method of preparing a complex, the method comprising:
providing a composition comprising a lactoferrin and a whey protein hydrolysate at a pH in a range of about 5, 5.5, or 5.8 to about 6, 6.5, or 7; and
mixing the composition, thereby providing the complex.

35. The method of claim 34, wherein the lactoferrin and the whey protein hydrolysate are each independently present in the composition in an amount of about 0.01%, 0.1%, 0.5%, or 1% to about 2% or 3% by weight of the composition.

36. The method of any one of claim 34 or 35, wherein the composition comprises the lactoferrin and the whey protein hydrolysate in a weight ratio of about 10:1 to about 1:10 (lactoferrin:whey protein hydrolysate), optionally wherein the composition comprises the lactoferrin and the whey protein hydrolysate in a weight ratio of about 1:1 or about 2:1 (lactoferrin:whey protein hydrolysate).

37. The method of any one of claims 34-36, wherein mixing the composition is carried out for about 1, 5, 10, or 15 minutes to about 20, 30, 40, 50, or 60 minutes, optionally at a temperature of about 20 °C to about 60 °C.

38. The method of any one of claims 34-37, further comprising isolating the complex from the composition, optionally wherein isolating the complex from the composition comprises centrifuging, drying, freeze-drying, and/or spray-drying the composition.

39. The method of any one of claims 34-38, wherein the total concentration of the lactoferrin and the whey protein hydrolysate in the composition is about 6% by weight of the composition or less (e.g., about 5%, 4%, 3%, 2%, 1%, or 0.5% by weight of the composition).

40. An article comprising a complex of any one of claims 1-25, a composition of any one of claims 26-33, and/or a complex prepared according to a method of any one of claims 34-39.

41. The article of claim 40, wherein the article is a food product (e.g., infant formula, a dairy product, etc.), nutritional supplement, therapeutic drink, and/or cosmetic.
